(19) United States
(12) Patent Application Publication
(10) Pub. No.: US 2009/0042957 A1
(43) Pub. Date: Feb. 12, 2009

(54) Substituted Oxyarenes

(75) Inventors: Peter Jeschke, Bergisch Gladbach (DE); Michael Muller, Dusseldorf (DE); Iris Escher, Ebsdorfergrund (DE); Michael E. Beck, Monheim (DE); Oliver Gaertzen, Koln (DE); Olga Malsam, Rosrath (DE); Karl-Josef Haack, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 11/793,764

(22) PCT Filed: Dec. 21, 2005
(86) PCT No.: PCT/EP05/13754

§ 371 Date: Mar. 28, 2008

(30) Foreign Application Priority Data
Dec. 24, 2004 (DE) 10 2004 062 542.5

(51) Int. Cl. A01N 43/80; C07D 413/12; C07C 251/34; C07C 65/21; C07C 69/76; C07D 261/04

(52) U.S. Cl. 514/378; 548/240; 564/265; 568/442; 560/61; 562/471

(57) ABSTRACT

The invention relates to compounds of the formula (I)

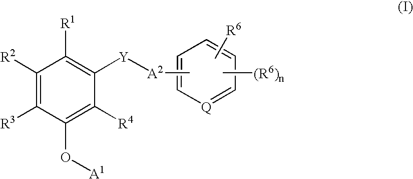

in which
A¹, A², R¹, R², R³, R⁴, R⁵ and Y are as defined in the description,
to processes and intermediates for their preparation and to their use for controlling pests.

The present invention provides novel substituted oxyarenes, processes for their preparation and their use as pesticides.

Substituted 5-benzyloxymethyl-4,5-dihydroisoxazoles are already known as herbicides for use in rice crops (cf. WO 02/19825, U.S. Pat. No. 4,983,210, U.S. Pat. No. 5,262,388, JP 09-143171); however, since their action is not always satisfactory, they have hitherto not attained any importance.

Substituted oxyarenes are furthermore described in WO 2004/099197.

This invention now provides novel substituted oxyarenes of the general formula (I)

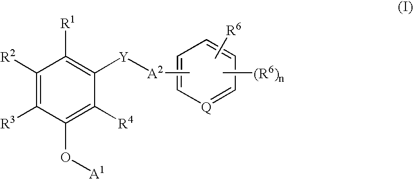

in which
n represents the number 0, 1 or 2,
A¹ represents one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃, —CH₂—CH═CClCH₃, where X represents halogen,
A² represents in each case straight-chain or branched alkanediyl or alkenediyl having in each case up to 8 carbon atoms which optionally contains at the beginning, at the end or within the carbon chain an oxygen atom, a sulfur atom or a grouping selected from the group consisting of SO, SO₂, NH and N(C₁-C₄-alkyl),
Q represents CH or N (nitrogen),
R¹ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino, alkylcarbonylamino or alkoximinoalkyl having in each case 1 to 10 carbon atoms in the alkyl groups, represents in each case optionally nitro-, hydroxyl-, amino-, cyano-, halogen-, C₁-C₆-alkyl-, C₁-C₆-haloalkyl-, C₁-C₆-alkoxy- or C₁-C₆-haloalkoxy-substituted aryloxy, arylthio or arylalkyl having in each case 6 or 10 carbon atoms in the aryl groups and, if appropriate, 1 to 4 carbon atoms in the alkyl moiety, represents in each case optionally nitro-, hydroxyl-, amino-, cyano-, halogen-, C₁-C₆-alkyl-, C₁-C₆-haloalkyl-, C₁-C₆-alkoxy- or C₁-C₆-haloalkoxy-substituted heterocyclyloxy or heterocyclylthio having in each case up to 10 carbon atoms, up to 4 nitrogen atoms and, if appropriate, one oxygen or sulfur atom, or represents the grouping —O-A¹, where A¹ is as defined above, or represents the grouping —N(R,R′), where R and R′ together represent straight-chain or branched alkanediyl having up to 8 carbon atoms which optionally contains at the beginning, at the end or within the carbon chain an oxygen atom, a sulfur atom or a grouping selected from the group consisting of SO, SO₂, NH and N(C₁-C₄-alkyl),
R² represents hydrogen, nitro, hydroxyl, amino, cyano, cyanato, thiocyanato, formyl, halogen, represents in each case optionally cyano-, halogen- or C₁-C₆-alkoxy-substituted alkyl, alkoxy, alkylthio, alkylsulfinyl, alkylsulfonyl, alkylamino, dialkylamino or alkylcarbonylamino having in each case 1 to 6 carbon atoms in the alkyl groups, represents C₁-C₆-alkyl-carbonyl, C₁-C₆-alkoxy-carbonyl, C₁-C₆-alkoximinoformyl, C₁-C₆-alkoximino-acetyl, or represents C₂-C₆-alkenyl or C₂-C₆-alkynyl,
R³ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁴ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁵ represents an alkenyl grouping or alkynyl grouping having in each case 2 to 6 carbon atoms or a cycloalkenyl grouping having 4 to 6 carbon atoms, each of which groupings contains at least one substituent from the group consisting of nitro, cyano, carboxyl, carbamoyl, hydroxyl, carbonyl (C═O), hydroximino (C═N—OH), C₁-C₆-alkoxy, C₁-C₆-alkoxy-carbonyl, C₁-C₆-alkylamino, di-(C₁-C₆-alkyl)-amino, C₁-C₆-alkylamino-carbonyl, C₁-C₆-alkoxy-carbonylamino, C₁-C₆-alkoxy-C₁-C₆-alkoxy, C₁-C₆-alkoxyimino, C₃-C₆-alkenyloxy, C₃-C₆-alkynyloxy, C₃-C₆-alkenyloxy-carbonyl, C₃-C₆-alkynyloxy-carbonyl, C₃-C₆-alkenyloxyimino, C₃-C₆-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl, or
R⁵ represents a grouping -A³-Z, where
   A³ represents a single bond or represents straight-chain or branched alkanediyl having 1 to 6 carbon atoms which is optionally substituted by halogen or C₃-C₆-cycloalkyl and
   Z represents monocyclic or bicyclic heteroaryl having up to 10 carbon atoms and at least one heteroatom from the group consisting of N (nitrogen, 1 to 5 N atoms), O (oxygen, 1 or 2 O atoms), sulfur (1 or 2 S atoms) and optionally as a replacement or additionally an SO or SO₂ grouping and optionally additionally a carbonyl grouping (C═O) and/or a thiocarbonyl grouping (C═S) as component of the heterocycle, which heteroaryl is optionally substituted by nitro, hydroxyl, mercapto, amino, formyl, cyano, carboxyl, carbamoyl, halogen, C₁-C₆-alkyl, C₁-C₆-hydroxyalkyl, C₁-C₆-haloalkyl, C₁-C₆-alkyl-carbonyl, C₁-C₆-haloalkyl-carbonyl, C₁-C₆-alkoxy, C₁-C₆-hydroxyalkoxy, C₁-C₆-haloalkoxy, C₁-C₆-alkoxy-carbonyl, C₁-C₆-haloalkoxy-carbonyl, C₁-C₆-alkoxy-C₁-C₆-alkyl, C₁-C₆-haloalkoxy-C₁-C₆-alkyl, C₁-C₂-alkylenedioxy, C₁-C₂-haloalkylenedioxy, C₁-C₆-alkoxyimino-C₁-C₆-alkyl, C₂-C₆-alkenyl, C₂-C₆-alkenyl-carbonyl, C₂-C₆-haloalkenyl, C₂-C₆-haloalkenyl-carbonyl, C₂-C₆-alkenyloxy-C₁-C₆-alkyl, C₂-C₆-haloalkenyloxy-C₁-C₆-alkyl, C₂-C₆-alkynyl, C₂-C₆-haloalkynyl, C₂-C₆-alkenyloxy, C₂-C₆-alkenyloxy-carbonyl, C₂-C₆-haloalkenyloxy, C₂-C₆-haloalkenyloxy-carbonyl, C₂-C₆-alkynyloxy, C₂-C₆-alkynyloxy-carbonyl, C₂-C₆-haloalkynyloxy, C₂-C₆-haloalkynyloxy-carbonyl, C₂-C₆-alkynyloxy-C₁-C₆-alkyl, C₂-C₆-haloalkynyloxy-C₁-C₆-alkyl, C₃-C₈-cycloalkyl, C₃-C₈-cycloalkyl-carbonyl, C₃-C₈-cycloalkyl-C₁-C₆-alkyl, C₃-C₈-cycloalkyl-C₁-C₆-alkyl-carbonyl, C₃-C₈-cycloalkyloxy, C₃-C₈-cycloalkyloxy-carbonyl, C₃-C₈-cycloalkyl-C₁-C₆-alkoxy, C₃-C₈-cycloalkyl-C₁-C₆-alkoxy-carbonyl, C₃-C₈-cycloalkyloxy-C₁-C₆-alkyl, C₃-C₈-cycloalkyloxy-C₁-C₆-alkoxy, C₃-C₈-cycloalkyl-C₁-C₆-alkoxy-C₁-C₆-alkyl, C₁-C₆-alkyl-carbonyl-C₁-C₆-alkyl, C₁-C₆-alkoxy-carbonyl-C₁-C₆-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₂-C₆-alkenylthio, C₂-C₆-haloalkenylthio, C₂-C₆-alkynylthio, C₃-C₆-cycloalkylthio, C₃-C₆-cycloalkyl-C₁-C₆-alkylthio, C₁-C₆-alkylamino, C₁-C₆-alkylamino-carbonyl, di-(C₁-C₆-alkyl)-amino, di-(C₁-C₆-alkyl)-amino-carbonyl, C₁-C₆-alkyl-carbonylamino, C₁-C₆-haloalkyl-carbonylamino, C₁-C₆-alkoxy-carbonylamino, C₁-C₆-alkyl-aminocarbonylamino, or by phenyl, phenyloxy, benzyl, benzyloxy, phenylamino, benzylamino (where in each case the phenyl groups are optionally substituted by nitro, hydroxyl, mercapto, amino, cyano, C₁-C₆-alkyl, C₁-C₆-haloalkyl, C₁-C₆-alkyl-carbonyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-haloalkenyloxy, C₂-C₆-alkynyloxy, C₁-C₆-alkylthio, C₁-C₆-alkylsulfinyl, C₁-C₆-alkylsulfonyl or C₁-C₆-alkoxy-carbonyl),
R⁶ represents nitro, hydroxyl, mercapto, amino, formyl, cyano, carboxyl, carbamoyl, halogen, C₁-C₆-alkyl, C₁-C₆-hydroxyalkyl, C₁-C₆-haloalkyl, C₁-C₆-alkyl-carbonyl, C₁-C₆-haloalkyl-carbonyl, C₁-C₆-alkoxy, C₁-C₆-hydroxyalkoxy, C₁-C₆-haloalkoxy, C₁-C₆-alkoxy-carbonyl, C₁-C₆-haloalkoxy-carbonyl, C₁-C₆-alkoxy-C₁-C₆-alkyl, C₁-C₆-haloalkoxy-C₁-C₆-alkyl, C₁-C₆-alkoxyimino-C₁-C₆-alkyl, C₂-C₆-alkenyl, C₂-C₆-alkenyl-carbonyl, C₂-C₆-haloalkenyl, C₂-C₆-haloalkenyl-carbonyl, C₂-C₆-alkenyloxy-C₁-C₆-alkyl, C₂-C₆-haloalkenyloxy-C₁-C₆-alkyl, C₂-C₆-alkynyl, C₂-C₆-haloalkynyl, C₂-C₆-alkenyloxy, C₂-C₆-alkenyloxy-carbonyl, C₂-C₆-haloalkenyloxy, C₂-C₆-haloalkenyloxy-carbonyl, C₂-C₆-alkynyloxy, C₂-C₆-alkynyloxy-carbonyl, C₂-C₆-haloalkynyloxy, C₂-C₆-haloalkynyloxy-carbonyl, C₂-C₆-alkynyloxy-C₁-C₆-alkyl, C₂-C₆-haloalkynyloxy-C₁-C₆-alkyl, C₁-C₆-alkyl-carbonyl-C₁-C₆-alkyl, C₁-C₆-alkoxy-carbonyl-C₁-C₆-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₂-C₆-alkenylthio, C₂-C₆-haloalkenylthio, C₂-C₆-alkynylthio, C₁-C₆-alkylamino, C₁-C₆-alkylamino-carbonyl, di-(C₁-C₆-alkyl)-amino, di-(C₁-C₆-alkyl)-amino-carbonyl, C₁-C₆-alkyl-carbonylamino, C₁-C₆-haloalkyl-carbonylamino, C₁-C₆-alkoxy-carbonylamino or C₁-C₆-alkyl-aminocarbonylamino, and
Y represents a five- or six-membered heterocyclic grouping having at least 2 carbon atoms, at least one nitrogen atom and optionally one oxygen or sulfur atom, which grouping is attached in two different positions to the adjacent groupings, represents in particular a heterocyclic grouping selected from the list below (here, the exocyclic bonds indicate in each case the linkages to the adjacent groupings according to the arrangement in formula (I)),

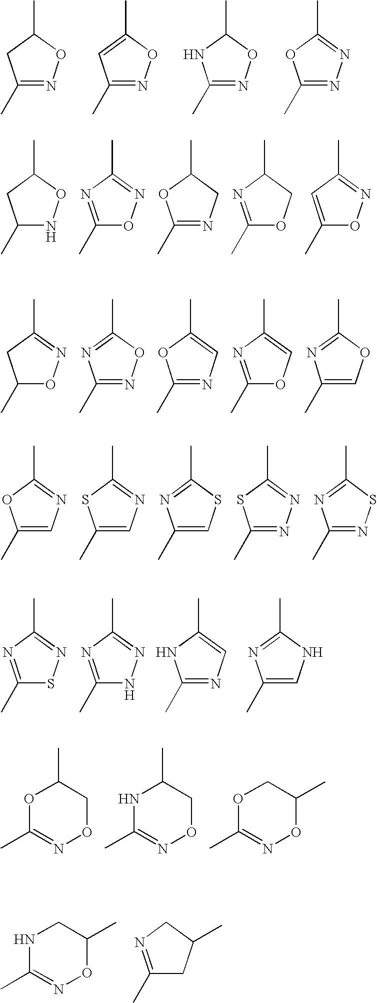

where these heterocyclic groupings may in each case optionally be substituted by one or two substituents from the group consisting of nitro, hydroxyl, amino, cyano, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio.

Depending on the nature of the substituents, the compounds of the formula (I) may, if appropriate, also be present as stereoisomers, i.e. as geometrical and/or as optical isomers or isomer mixtures of varying composition. The present invention provides both the pure stereoisomers and any mixtures of these isomers, even if generally only the compounds of the formula (I) are referred to.

The invention also provides the salt-like derivatives formed from compounds of the formula (I) by reaction with basic or acidic compounds.

Preferred substituents or ranges of the radicals present in the formulae given above and below are defined below.
A¹ preferably represents one of the groupings below:
   —CH₂—CH═CCl₂, —CH₂—CH═CBr₂, —CH₂—CH═CClF, —CH₂—CH═CBrCl, —CH₂—CH═CBrF.
A² preferably represents in each case straight-chain or branched alkanediyl or alkenediyl having in each case up to 4 carbon atoms which optionally contains at the beginning, at the end or within the carbon chain an oxygen atom, a sulfur atom or a grouping selected from the group consisting of SO, SO₂, NH and N(C₁-C₃-alkyl).
Q preferably represents CH or N (nitrogen).
R¹ preferably represents hydrogen, nitro, hydroxyl, amino, cyano, halogen, represents in each case optionally cyano-, halogen- or C₁-C₅-alkoxy-substituted alkyl, alkoxy, alkylthio, alkylamino, dialkylamino, alkylcarbonylamino or alkoximinoalkyl having in each case 1 to 8 carbon atoms in the alkyl groups, represents in each case optionally nitro-, hydroxyl-, amino-, cyano-, halogen-, C₁-C₅-alkyl-, C₁-C₈-haloalkyl-, C₁-C₅-alkoxy- or C₁-C₅-haloalkoxy-substituted aryloxy, arylthio or arylalkyl having in each case 6 or 10 carbon atoms in the aryl groups and, if appropriate, 1 to 3 carbon atoms in the alkyl moiety, represents in each case optionally nitro-, hydroxyl-, amino-, cyano-, halogen-, C₁-C₅-alkyl-, C₁-C₈-haloalkyl-, C₁-C₅-alkoxy- or C₁-C₅-haloalkoxy-substituted heterocyclyloxy or heterocyclylthio having in each case up to 9 carbon atoms, 1 to 4 nitrogen atoms and/or one oxygen or sulfur atom, or represents the grouping —O-A¹, where A¹ is as defined above, or represents the grouping —N(R,R′), where R and R′ together represent straight-chain or branched alkanediyl having up to 6 carbon atoms which optionally contains at the beginning, at the end or within the carbon chain an oxygen atom, a sulfur atom or a grouping selected from the group consisting of SO, SO₂, NH and N(C₁-C₃-alkyl).
R² preferably represents hydrogen, nitro, cyano, cyanato, thiocyanato, formyl, halogen, represents in each case optionally cyano-, halogen- or C₁-C₅-alkoxy-substituted alkyl, alkoxy, alkylthio, alkylamino, dialkylamino or alkylcarbonylamino having in each case 1 to 5 carbon atoms in the alkyl groups, represents C₁-C₅-alkyl-carbonyl, C₁-C₅-alkoxy-carbonyl, C₁-C₅-alkoximinoformyl, C₁-C₅-alkoximino-acetyl, or represents C₂-C₅-alkenyl or C₂-C₈-alkynyl.
R³ preferably represents hydrogen, nitro, halogen, represents in each case optionally cyano-, halogen- or C₁-C₅-alkoxy-substituted alkyl, alkoxy, alkylthio or alkylamino having in each case 1 to 5 carbon atoms in the alkyl groups.
R⁴ preferably represents hydrogen, nitro, halogen, represents in each case optionally cyano-, halogen- or C₁-C₅-alkoxy-substituted alkyl, alkoxy, alkylthio or alkylamino having in each case 1 to 5 carbon atoms in the alkyl groups.
R⁵ preferably represents an alkenyl grouping or alkynyl grouping having in each case 2 to 5 carbon atoms or a cycloalkenyl grouping having 4 to 6 carbon atoms, each of which groupings contains at least one substituent from the group consisting of nitro, cyano, carboxyl, carbamoyl, hydroxyl, carbonyl (C═O), hydroximino (C═N—OH), C₁-C₅-alkoxy, C₁-C₅-alkoxy-carbonyl, C₁-C₅-alkylamino, di-(C₁-C₄-alkyl)-amino, C₁-C₅-alkylamino-carbonyl, C₁-C₅-alkoxy-carbonylamino, C₁-C₅-alkoxy-C₁-C₅-alkoxy, C₁-C₅-alkoxyimino, C₃-C₅-alkenyloxy, C₃-C₅-alkynyloxy, C₃-C₅-alkenyloxy-carbonyl, C₃-C₅-alkynyloxy-carbonyl, C₃-C₅-alkenyloxyimino, C₃-C₅-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl.
R⁵ preferably furthermore represents a grouping -A³-Z, where
   A³ represents a single bond or represents straight-chain or branched alkanediyl having 1 to 5 carbon atoms which is optionally substituted by halogen or C₃-C₆-cycloalkyl and
   Z represents monocyclic or bicyclic heteroaryl having up to 9 carbon atoms and at least one heteroatom from the group consisting of N (nitrogen, 1 to 5 N atoms), O (oxygen, 1 or 2 O atoms), sulfur (1 or 2 S atoms) and optionally as a replacement or additionally an SO or SO₂ grouping and optionally additionally a carbonyl grouping (C═O) and/or a thiocarbonyl grouping (C═S) as component of the heterocycle, which heteroaryl is optionally substituted by nitro, hydroxyl, mercapto, amino, formyl, cyano, carboxyl, carbamoyl, halogen, C₁-C₅-alkyl, C₁-C₅-hydroxyalkyl, C₁-C₅-haloalkyl, C₁-C₅-alkyl-carbonyl, C₁-C₈-haloalkyl-carbonyl, C₁-C₈-alkoxy, C₁-C₅-hydroxyalkoxy, C₁-C₅-haloalkoxy, C₁-C₅-alkoxy-carbonyl, C₁-C₅-haloalkoxy-carbonyl, C₁-C₅-alkoxy-C₁-C₅-alkyl, C₁-C₅-haloalkoxy-C₁-C₅-alkyl, C₁-C₂-alkylenedioxy, C₁-C₂-haloalkylenedioxy, C₁-C₅-alkoxyimino-C₁-C₅-alkyl, C₂-C₅-alkenyl, C₂-C₅-alkenyl-carbonyl, C₂-C₅-haloalkenyl, C₂-C₅-haloalkenyl-carbonyl, C₃-C₅-alkenyloxy-C₁-C₆-alkyl, C₃-C₅-haloalkenyloxy-C₁-C₅-alkyl, C₂-C₅-alkynyl, C₂-C₅-haloalkynyl, C₃-C₅-alkenyloxy, C₃-C₅-alkenyloxy-carbonyl, C₃-C₅-haloalkenyloxy, C₃-C₅-haloalkenyloxy-carbonyl, C₃-C₅-alkynyloxy, C₃-C₅-alkynyloxy-carbonyl, C₃-C₅-haloalkynyloxy, C₃-C₈-haloalkynyloxy-carbonyl, C₃-C₅-alkynyloxy-C₁-C₅-alkyl, C₃-C₅-haloalkynyloxy-C₁-C₅-alkyl, C₃-C₇-cycloalkyl, C₃-C₇-cycloalkyl-carbonyl, C₃-C₇-cycloalkyl-C₁-C₅-alkyl, C₃-C₇-cycloalkyl-C₁-C₅-alkyl-carbonyl, C₃-C₇-cycloalkyloxy, C₃-C₇-cycloalkyloxy-carbonyl, C₃-C₇-cycloalkyl-C₁-C₅-alkoxy, C₃-C₇-cycloalkyl-C₁-C₅-alkoxy-carbonyl, C₃-C₇-cycloalkyloxy-C₁-C₅-alkyl, C₃-C₇-cycloalkyloxy-C₁-C₅-alkoxy, C₃-C₇-cycloalkyl-C₁-C₈-alkoxy-C₁-C₅-alkyl, C₁-C₅-alkyl-carbonyl-C₁-C₅-alkyl, C₁-C₅-alkoxy-carbonyl-C₁-C₅-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, C₂-C₅-alkenylthio, C₂-C₈-haloalkenylthio, C₂-C₅-alkynylthio, C₃-C₆-cycloalkylthio, C₃-C₆-cycloalkyl-C₁-C₅-alkylthio, C₁-C₅-alkylamino, C₁-C₅-alkylamino-carbonyl, di-(C₁-C₄-alkyl)-amino, di-(C₁-C₄-alkyl)-amino-carbonyl, C₁-C₅-alkyl-carbonylamino, C₁-C₅-haloalkyl-carbonylamino, C₁-C₅-alkoxy-carbonylamino, C₁-C₅-alkyl-aminocarbonylamino, or by phenyl, phenyloxy, benzyl, benzyloxy, phenylamino, benzylamino (where the phenyl groups are in each case optionally substituted by nitro, hydroxyl, mercapto, amino, cyano, C₁-C₅-alkyl, C₁-C₅-haloalkyl, C₁-C₅-alkyl-carbonyl, C₂-C₅-alkenyl, C₂-C₅-haloalkenyl, C₂-C₅-alkynyl, C₂-C₅-haloalkynyl, C₁-C₅-alkoxy, C₁-C₅-haloalkoxy, C₂-C₅-alkenyloxy, C₂-C₅-haloalkenyloxy, C₂-C₅-alkynyloxy, C₁-C₅-alkylthio, C₁-C₅-alkylsulfinyl, C₁-C₅-alkylsulfonyl or C₁-C₅-alkoxy-carbonyl).
R⁶ preferably represents nitro, hydroxyl, mercapto, amino, formyl, cyano, carboxyl, carbamoyl, halogen, C₁-C₅-alkyl, C₁-C₅-hydroxyalkyl, C₁-C₅-haloalkyl, C₁-C₅-alkyl-carbonyl, C₁-C₅-haloalkyl-carbonyl, C₁-C₅-alkoxy, C₁-C₅-hydroxyalkoxy, C₁-C₅-haloalkoxy, C₁-C₅-alkoxy-carbonyl, C₁-C₅-haloalkoxy-carbonyl, C₁-C₅-alkoxy-C₁-C₅-alkyl, C₁-C₅-haloalkoxy-C₁-C₅-alkyl, C₁-C₅-alkoxyimino-C₁-C₅-alkyl, C₂-C₅-alkenyl, C₂-C₅-alkenyl-carbonyl, C₂-C₅-haloalkenyl, C₂-C₅-haloalkenyl-carbonyl, C₂-C₅-alkenyloxy-C₁-C₅-alkyl, C₂-C₅-haloalkenyloxy-C₁-C₅-alkyl, C₂-C₅-alkynyl, C₂-C₅-haloalkynyl, C₂-C₅-alkenyloxy, C₃-C₅-alkenyloxy-carbonyl, C₃-C₅-haloalkenyloxy, C₃-C₅-haloalkenyloxy-carbonyl, C₃-C₅-alkynyloxy, C₃-C₅-alkynyloxy-carbonyl, C₃-C₅-haloalkynyloxy, C₃-C₅-haloalkynyloxy-carbonyl, C₃-C₅-alkynyloxy-C₁-C₆-alkyl, C₃-C₅-haloalkynyloxy-C₁-C₅-alkyl, C₁-C₅-alkyl-carbonyl-C₁-C₅-alkyl, C₁-C₅-alkoxy-carbonyl-C₁-C₅-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, C₃-C₅-alkenylthio, C₃-C₅-haloalkenylthio, C₃-C₅-alkynylthio, C₁-C₅-alkylamino, C₁-C₅-alkylamino-carbonyl, di-(C₁-C₄-alkyl)-amino, di-(C₁-C₄-alkyl)-amino-carbonyl, C₁-C₅-alkyl-carbonylamino, C₁-C₅-haloalkyl-carbonylamino, C₁-C₅-alkoxy-carbonylamino or C₁-C₅-alkyl-aminocarbonylamino.
Y preferably represents a heterocyclic grouping which is attached in two different positions to the adjacent groupings and selected from the following list (here, the exocyclic bonds indicate in each case the linkages to the adjacent groupings according to the arrangement in formula (I)),

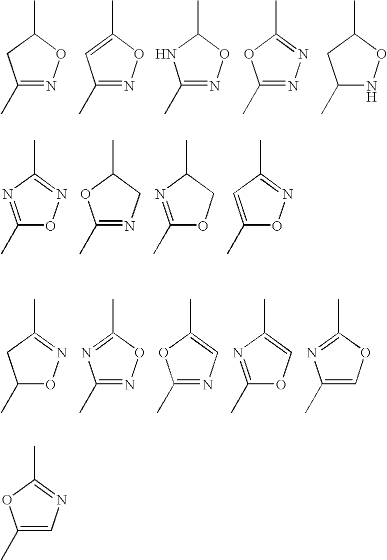

where these heterocyclic groupings may in each case optionally be substituted by one or two substituents from the group consisting of nitro, hydroxyl, amino, cyano, halogen, C₁-C₅-alkyl, C₁-C₅-haloalkyl, C₁-C₅-alkoxy, C₁-C₅-haloalkoxy, C₁-C₅-alkylthio, C₁-C₅-haloalkylthio.
n particularly preferably represents the number 0 or 1.
A¹ particularly preferably represents one of the groupings below:
   —CH₂—CH═CCl₂, —CH₂—CH═CBr₂, —CH₂—CH═CBrCl.
A² particularly preferably represents one of the alkanediyl groupings listed below
   —CH₂—, —CH₂CH₂—, —CH(CH₃)—CH₂—, —CH₂CH(CH₃)—, —CH₂CH₂CH₂—, —CH(CH₃)CH₂CH₂—, —CH₂CH(CH₃)CH₂—, —CH₂CH₂CH(CH₃)—, —CH₂CH₂CH₂CH₂—, —CH₂CH₂CH₂CH₂CH₂—,
   each of which optionally contains at the beginning, at the end or within the carbon chain an oxygen atom, a sulfur atom or a grouping selected from the group consisting of SO, SO₂, NH and N(methyl).
Q particularly preferably represents CH or N (nitrogen).
R¹ particularly preferably represents hydrogen, nitro, hydroxyl, amino, cyano, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, dipropylamino, acetylamino, propionylamino, n- or i-butyroylamino, methoximinomethyl, ethoximinomethyl, methoximinoethyl or ethoximinoethyl, represents in each case optionally nitro-, hydroxyl-, amino-, cyano-, fluorine-, chlorine-, bromine-, iodine-, methyl-, ethyl-, n- or i-propyl-, n-, i-, s- or t-butyl-, difluoromethyl-, trifluoromethyl-, chlorodifluoromethyl-, fluoroethyl-, difluoroethyl-, trifluoroethyl-, chloroethyl-, dichloroethyl-, trichloroethyl-, methoxy-, ethoxy-, n- or i-propoxy, n-, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted phenoxy, naphthyloxy, phenylthio, naphthylthio, benzyl or phenylethyl, represents in each case optionally nitro-, hydroxyl-, amino-, cyano-, fluorine-, chlorine-, bromine-, iodine-, methyl-, ethyl-, n- or i-propyl-, n-, i-, s- or t-butyl-, difluoromethyl-, trifluoromethyl-, chlorodifluoromethyl-, fluoroethyl-, difluoroethyl, trifluoroethyl, chloroethyl-, dichloroethyl-, trichloroethyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted heterocyclyloxy or heterocyclylthio having in each case up to 9 carbon atoms, 1 to 4 nitrogen atoms and/or one oxygen or sulfur atom, or represents the grouping —O-A¹, where A¹ is as defined above, or represents the grouping —N(R,R′) where R and R′ together with the N atom to which they are attached represent pyrrolidinyl, piperidinyl, morpholinyl or piperazinyl, each of which is optionally mono- or disubstituted by methyl and/or ethyl.
R² particularly preferably represents hydrogen, nitro, cyano, cyanato, thiocyanato, formyl, fluorine, chlorine, bromine, iodine, represents in each case optionally cyano-, fluorine, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, acetylamino, propionylamino, n- or i-butyroylamino, acetyl, propionyl, n- or i-butyroyl, methoxycarbonyl, ethoxycarbonyl, n- or i-propoxycarbonyl, methoximinoformyl, ethoximinoformyl, methoximinoacetyl or ethoximinoacetyl.
R³ particularly preferably represents hydrogen, nitro, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino.
R⁴ particularly preferably represents hydrogen, nitro, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino.
R⁵ particularly preferably represents an ethenyl, propenyl, butenyl, pentenyl, ethynyl, propynyl, butynyl, pentynyl, cyclobutenyl, cyclopentenyl or cyclohexenyl grouping, each of which contains at least one substituent from the group consisting of nitro, cyano, carboxyl, carbamoyl, hydroxyl, carbonyl (C═O), hydroximino (C═N—OH), methoxy, ethoxy, n- or i-propoxy, n-, i-, s- or t-butoxy, methoxycarbonyl, ethoxycarbonyl, n- or ipropoxycarbonyl, n-, i-, s- or t-butoxycarbonyl, methylamino, ethylamino, n- or i-propylamino, n-, i-, s- or t-butyl amino, dimethyl amino, diethylamino, dipropylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methoxymethoxy, ethoxymethoxy, n- or ipropoxymethoxy, n-, i-, s- or t-butoxymethoxy, methoxyethoxy, ethoxyethoxy, n- or ipropoxyethoxy, methoxypropoxy, ethoxypropoxy, methoxyimino, ethoxyimino, n- or ipropoxyimino, n-, i-, s- or t-butoxyimino, propenyloxy, butenyloxy, propynyloxy, butynyloxy, propenyloxycarbonyl, butenyloxycarbonyl, propynyloxycarbonyl, butynyloxycarbonyl, propenyloxyimino, butenyloxyimino, propynyloxyimino, butynyloxyimino, cyclopropyl, cyclobutyl, cyclopentyl, cyclohexyl, furyl, benzofuryl, thienyl, benzothienyl, isoxazolyl, benzisoxazolyl, pyrazolyl, pyridinyl, pyrimidinyl, pyrazinyl, pyridazinyl.
R⁵ particularly preferably furthermore represents a grouping -A³-Z, where
   A³ represents a single bond or represents in each case optionally fluorine-, chlorine-, bromine-, cyclopropyl-, cyclobutyl-, cyclopentyl- or cyclohexyl-substituted methylene, ethane-1,1-diyl (ethylidene), ethane-1,2-diyl (dimethylene), propane-1,1-diyl (propylidene), propane-1,2-diyl, propane-1,3-diyl (trimethylene), butane-1,1-diyl (butylidene) or butane-1,4-diyl (tetramethylene) and
   Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, hydroxy-n-propyl, hydroxy-1-propyl, fluoromethyl, chloromethyl, bromomethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-i-propyl, difluoro-i-propyl, trifluoro-i-propyl, tetrafluoro-i-propyl, pentafluoro-i-propyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, hydroxyethoxy, hydroxypropoxy, hydroxybutoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, difluoropropoxy, trifluoropropoxy, methoxycarbonyl, ethoxycarbonyl, n- or ipropoxycarbonyl, n-, i-, s- or t-butoxycarbonyl, fluoroethoxycarbonyl, chloroethoxycarbonyl, methoxymethyl, ethoxymethyl, n- or i-propoxymethyl, n-, i-, s- or t-butoxymethyl, methoxyethyl, ethoxyethyl, n- or i-propoxyethyl, fluoromethoxymethyl, difluoromethoxymethyl, fluoromethoxyethyl, difluoromethoxyethyl, methylenedioxy, dimethylenedioxy, difluoromethylenedioxy, difluorodimethylenedioxy, trifluorodimethylenedioxy, methoxyiminomethyl, ethoxyiminomethyl, n- or ipropoxyiminomethyl, n-, i-, s- or t-butoxyiminomethyl, methoxyiminoethyl, ethoxyiminoethyl, n- or i-propoxyiminoethyl, ethenyl, propenyl, butenyl, ethenylcarbonyl, propenylcarbonyl, butenylcarbonyl, fluoroethenyl, chloroethenyl, difluoroethenyl, dichloroethenyl, trifluoroethenyl, trichloroethenyl, fluoroethenylcarbonyl, chloroethenylcarbonyl, difluoroethenylcarbonyl, dichloroethenylcarbonyl, trifluoroethenylcarbonyl, trichloroethenylcarbonyl, propenyloxymethyl, butenyloxymethyl, propenyloxyethyl, butenyloxyethyl, fluoropropenyloxymethyl, chloropropenyloxymethyl, difluoropropenyloxymethyl, dichloropropenyloxymethyl, fluoropropenyloxyethyl, chloropropenyloxyethyl, difluoropropenyloxyethyl, dichloropropenyloxyethyl, ethynyl, propynyl, butynyl, fluoropropynyl, chloropropynyl, fluorobutynyl, chlorobutynyl, propenyloxy, butenyloxy, fluoropropenyloxy, chloropropenyloxy, fluorobutenyloxy, chlorobutenyloxy, propenyloxycarbonyl, butenyloxycarbonyl, fluoropropenyloxycarbonyl, chloropropenyloxycarbonyl, fluorobutenyloxycarbonyl, chlorobutenyloxycarbonyl, propynyloxy, butynyloxy, propynyloxycarbonyl, butynyloxycarbonyl, fluoropropynyloxy, chloropropynyloxy, fluorobutynyloxy, chlorobutynyloxy, fluoropropynyloxycarbonyl, chloropropynyloxycarbonyl, fluorobutynyloxycarbonyl, chlorobutynyloxycarbonyl, propynyloxymethyl, butynyloxymethyl, propynyloxyethyl, butynyloxyethyl, fluoropropynyloxymethyl, chloropropynyloxymethyl, fluorobutynyloxymethyl, chlorobutynyloxymethyl, fluoropropynyloxyethyl, chloropropynyloxyethyl, fluorobutynyloxyethyl, chlorobutynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, n-, i-, s- or t-butylthio, difluoromethylthio, trifluoromethylthio, chlorodifluoromethylthio, methylsulfinyl, ethylsulfinyl, n- or i-propylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, dimethylamino, diethylamino, dipropylamino, dibutylamino, dimethylaminocarbonyl, diethylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, methoxycarbonylamino, ethoxycarbonylamino, n- or i-propoxycarbonylamino, n-, i-, s- or t-butoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, acetyl, propionyl, n- or i-butyroyl, ethenyl, propenyl, butenyl, fluoroethenyl, chloroethenyl, difluoroethenyl, dichloroethenyl, trifluoroethenyl, trichloroethenyl, ethynyl, propynyl, butynyl, fluoropropynyl, chloropropynyl, fluorobutynyl, chlorobutynyl, methoxy, ethoxy, n- or i-propoxy, n-, i-, s- or t-butoxy, fluoromethoxy, difluoromethoxy, trifluoromethoxy, fluoroethoxy, difluoroethoxy, trifluoroethoxy, propenyloxy, butenyloxy, fluoropropenyloxy, chloropropenyloxy, fluorobutenyloxy, chlorobutenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or i-propoxycarbonyl, n-, i-, s- or t-butoxycarbonyl).
R⁶ particularly preferably represents nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, bromomethyl, difluoromethyl, dichloromethyl, trifluoromethyl, trichloromethyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, fluoromethoxy, difluoromethoxy, trifluoromethoxy, fluoroethoxy, chloroethoxy, difluoroethoxy, dichloroethoxy, trifluoroethoxy, fluoropropoxy, hydroxyethoxy, hydroxypropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, n-, i-, s- or t-butoxymethyl, methoxyethyl, ethoxyethyl, n- or i-propoxyethyl, fluoromethoxymethyl, difluoromethoxymethyl, fluoromethoxyethyl, difluoromethoxyethyl, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, butenyl, ethenylcarbonyl, propenylcarbonyl, butenylcarbonyl, fluoroethenyl, chloroethenyl, difluoroethenyl, dichloroethenyl, trifluoroethenyl, fluoroethenylcarbonyl, chloroethenylcarbonyl, difluoroethenylcarbonyl, dichloroethenylcarbonyl, trifluoroethenylcarbonyl, propenyloxymethyl, butenyloxymethyl, propenyloxyethyl, butenyloxyethyl, fluoropropenyloxymethyl, chloropropenyloxymethyl, fluorobutenyloxymethyl, chlorobutenyloxymethyl, fluoropropenyloxyethyl, chloropropenyloxyethyl, fluorobutenyloxyethyl, chlorobutenyloxyethyl, ethynyl, propynyl, butynyl, fluoropropynyl, chloropropynyl, fluorobutynyl, chlorobutynyl, propenyloxy, butenyloxy, propenyloxycarbonyl, butenyloxycarbonyl, fluoropropenyloxy, chloropropenyloxy, fluorobutenyloxy, chlorobutenyloxy, fluoropropenyloxycarbonyl, chloropropenyloxycarbonyl, fluorobutenyloxycarbonyl, chlorobutenyloxycarbonyl, propynyloxy, butynyloxy, propynyloxycarbonyl, butynyloxycarbonyl, fluoropropynyloxy, chloropropynyloxy, fluorobutynyloxy, chlorobutynyloxy, fluoropropynyloxycarbonyl, chloropropynyloxycarbonyl, fluorobutynyloxycarbonyl, chlorobutynyloxycarbonyl, propynyloxymethyl, butynyloxymethyl, propynyloxyethyl, butynyloxyethyl, fluoropropynyloxymethyl, chloropropynyloxymethyl, fluorobutynyloxymethyl, chlorobutynyloxymethyl, acetylmethyl, propionylmethyl, n- or i-butyroylmethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, n-, i-, s- or t-butylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, dimethylamino, diethylamino, dipropylamino, dimethylaminocarbonyl, diethylaminocarbonyl, dipropylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, fluoropropionylamino, chloropropionylamino, methoxycarbonylamino, ethoxycarbonylamino, n- or i-propoxycarbonylamino, methylaminocarbonylamino, ethylaminocarbonylamino, n- or i-propylaminocarbonylamino.

Y particularly preferably represents a heterocyclic grouping which is attached in two different positions to the adjacent groupings and selected from the list below (here, the exocyclic bonds indicate in each case the linkages to the adjacent groupings according to the arrangement in formula (I)),

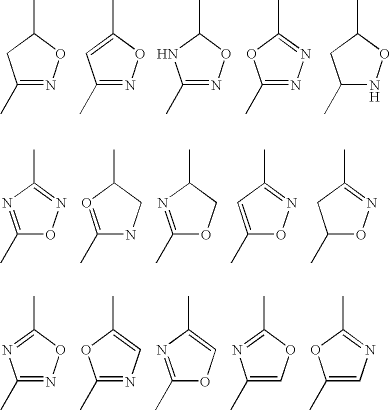

where these heterocyclic groupings may each optionally be substituted by one or two substituents from the group consisting of nitro, hydroxyl, amino, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, n-, i-, s- or t-butylthio, difluoromethylthio, trifluoromethylthio or chlorodifluoromethylthio.
A¹ very particularly preferably represents the grouping —CH₂—CH═CCl₂.
A² very particularly preferably represents one of the groupings listed below:
   —CH₂O—, —CH₂CH₂O—, —CH₂CH₂CH₂O—, —CH₂CH₂CH₂CH₂O—.
Q very particularly preferably represents CH.
R¹ very particularly preferably represents hydrogen, nitro, hydroxyl, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino, dimethylamino, represents in each case optionally nitro-, hydroxyl-, cyano-, fluorine-, chlorine-, bromine-, methyl-, ethyl-, n- or i-propyl-, n-, i-, s- or t-butyl-, difluoromethyl-, trifluoromethyl-, chlorodifluoromethyl-, fluoroethyl-, difluoroethyl-, trifluoroethyl-, chloroethyl-, dichloroethyl-, trichloroethyl-, methoxy-, ethoxy-, n- or i-propoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted phenoxy, phenylthio, benzyl or phenylethyl, or represents the grouping —O-A¹, where A¹ has one of the meanings given above.
R² very particularly preferably represents hydrogen, cyano, fluorine, chlorine, bromine, methyl, ethyl, methoxy or ethoxy.
R³ very particularly preferably represents hydrogen, cyano, fluorine, chlorine, bromine, methyl, ethyl, methoxy or ethoxy.
R⁴ very particularly preferably represents hydrogen, cyano, fluorine, chlorine or bromine.
R⁵ very particularly preferably represents an ethenyl, propenyl, butenyl, pentenyl, ethynyl, propynyl, butynyl, pentynyl, cyclobutenyl, cyclopentenyl or cyclohexenyl grouping, each of which contains at least one substituent from the group consisting of nitro, cyano, hydroxyl, carbonyl (C═O), hydroximino (C═N—OH), methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, dimethylamino, diethylamino, methylaminocarbonyl, ethylaminocarbonyl, n- or i-propylaminocarbonyl, methoxycarbonylamino, ethoxycarbonylamino, n- or i-propoxycarbonylamino, n-, i-, s- or t-butoxycarbonylamino, methoxymethoxy, ethoxymethoxy, n- or i-propoxymethoxy, methoxyethoxy, ethoxyethoxy, n- or i-propoxyethoxy, methoxypropoxy, ethoxypropoxy, methoxyimino, ethoxyimino, n- or i-propoxyimino, propenyloxy, butenyloxy, propynyloxy, butynyloxy, propenyloxycarbonyl, butenyloxycarbonyl, propynyloxycarbonyl, butynyloxycarbonyl, propenyloxyimino, butenyloxyimino, propynyloxyimino, butynyloxyimino, cyclopropyl, cyclobutyl, cyclopentyl, cyclohexyl, furyl, benzofuryl, thienyl, pyridinyl.
R⁵ very particularly preferably furthermore represents a grouping -A³-Z, where
   A³ represents a single bond or represents in each case optionally fluorine-, chlorine-, cyclopropyl-, cyclobutyl-, cyclopentyl- or cyclohexyl-substituted methylene, ethane-1,1-diyl (ethylidene), ethane-1,2-diyl (dimethylene), propane-1,1-diyl (propylidene), propane-1,2-diyl or propane-1,3-diyl (trimethylene) and
   Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-i-propyl, difluoro-i-propyl, trifluoro-i-propyl, tetrafluoro-i-propyl, pentafluoro-i-propyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, hydroxyethoxy, hydroxypropoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, n-, i-, s- or t-butoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, fluoromethoxymethyl, difluoromethoxymethyl, fluoromethoxyethyl, difluoromethoxyethyl, methylenedioxy, dimethylenedioxy, difluoromethylenedioxy, difluorodimethylenedioxy, trifluorodimethylenedioxy, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, butenyl, propenylcarbonyl, butenylcarbonyl, fluoroethenyl, chloroethenyl, difluoroethenyl, dichloroethenyl, trifluoroethenyl, trichloroethenyl, propenyloxymethyl, butenyloxymethyl, propenyloxyethyl, butenyloxyethyl, fluoropropenyloxymethyl, chloropropenyloxymethyl, difluoropropenyloxymethyl, dichloropropenyloxymethyl, fluoropropenyloxyethyl, chloropropenyloxyethyl, difluoropropenyloxyethyl, dichloropropenyloxyethyl, ethynyl, propynyl, butynyl, fluoropropynyl, chloropropynyl, fluorobutynyl, chlorobutynyl, propenyloxy, butenyloxy, fluoropropenyloxy, chloropropenyloxy, fluorobutenyloxy, chlorobutenyloxy, propenyloxycarbonyl, butenyloxycarbonyl, propynyloxy, butynyloxy, propynyloxycarbonyl, butynyloxycarbonyl, propynyloxymethyl, butynyloxymethyl, propynyloxyethyl, butynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, methoxycarbonylamino, ethoxycarbonylamino, n- or i-propoxycarbonylamino, n-, i-, s- or t-butoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, acetyl, propionyl, ethenyl, propenyl, ethynyl, propynyl, methoxy, ethoxy, n- or i-propoxy, fluoromethoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or i-propoxycarbonyl).
R⁶ very particularly preferably represents nitro, hydroxyl, mercapto, amino, formyl, cyano, carboxyl, carbamoyl, fluorine, chlorine, bromine, methyl, ethyl, n- or i-propyl, fluoromethyl, chloromethyl, bromomethyl, difluoromethyl, dichloromethyl, trifluoromethyl, trichloromethyl, acetyl, propionyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, fluoromethoxy, difluoromethoxy, trifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, fluoroethenyl, chloroethenyl, difluoroethenyl, dichloroethenyl, trifluoroethenyl, ethynyl, propynyl, butynyl, propenyloxy, butenyloxy, propenyloxycarbonyl, butenyloxycarbonyl, fluoropropenyloxy, chloropropenyloxy, fluorobutenyloxy, chlorobutenyloxy, propynyloxy, butynyloxy, propynyloxycarbonyl, butynyloxycarbonyl, propynyloxymethyl, butynyloxymethyl, propynyloxyethyl, butynyloxyethyl, acetylmethyl, propionylmethyl, n- or i-butyroylmethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, propynylthio, butynylthio, methylamino, ethylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, methoxycarbonylamino, ethoxycarbonylamino, n- or i-propoxycarbonylamino, methylaminocarbonylamino, ethylaminocarbonylamino, n- or i-propylaminocarbonylamino,
Y very particularly preferably represents one of the heterocyclic groupings below (here, the exocyclic bonds indicate in each case the linkages to the adjacent groupings according to the arrangement in formula (I)),

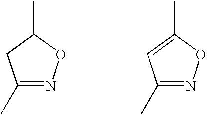

where each of these heterocyclic groupings may optionally be substituted by one or two substituents from the group consisting of nitro, hydroxyl, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, difluoromethylthio, trifluoromethylthio or chlorodifluoromethylthio.
R¹ most preferably represents hydrogen, nitro, hydroxyl, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino or dimethylamino.
R² most preferably represents hydrogen, fluorine, chlorine or bromine.
R⁵ most preferably represents a grouping -A³-Z, where
   A³ represents a single bond or represents methylene and
   Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, very particularly however tetrazolyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-1-propyl, difluoro-i-propyl, trifluoro-i-propyl, tetrafluoro-i-propyl, pentafluoro-1-propyl, methoxy, ethoxy, n- or i-propoxy, hydroxyethoxy, hydroxypropoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, ethenyl, propenyl, butenyl, fluoroethenyl, chloroethenyl, difluoroethenyl, dichloroethenyl, trifluoroethenyl, trichloroethenyl, propenyloxymethyl, butenyloxymethyl, propenyloxyethyl, butenyloxyethyl, ethynyl, propynyl, butynyl, propenyloxy, butenyloxy, fluoropropenyloxy, chloropropenyloxy, fluorobutenyloxy, chlorobutenyloxy, propenyloxycarbonyl, butenyloxycarbonyl, propynyloxy, butynyloxy, propynyloxycarbonyl, butynyloxycarbonyl, propynyloxymethyl, butynyloxymethyl, propynyloxyethyl, butynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, n- or i-butyroylamino, methoxycarbonylamino, ethoxycarbonylamino, n- or i-propoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, methoxy, ethoxy, n- or i-propoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or i-propoxycarbonyl).
Y most preferably represents one of the heterocyclic groupings below (here, the exocyclic bonds indicate in each case the linkages to the adjacent groupings according to the arrangement in formula (I))

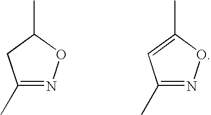

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

Preference according to the invention is given to the compounds of the formula (I) which contain a combination of the meanings listed above as being preferred.

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Most preference according to the invention is given to the compounds of the formula (I) which contain a combination of the meanings listed above as being most preferred.

In the radical definitions given above and below, hydrocarbon radicals, such as alkyl, are in each case straight-chain or branched as far as this is possible—including in combination with heteroatoms, such as in alkoxy.

The novel substituted oxyarenes of the general formula (I) have interesting biological properties. In particular, they are distinguished by strong arthropodicidal (insecticidal and acaricidal) and also nematicidal activity and can be used in agriculture, in forests, in the protection of stored products and in the protection of materials and also in the field of hygiene.

The novel substituted oxyarenes of the general formula (I) are obtained when substituted benzaldoximes of the general formula (II)

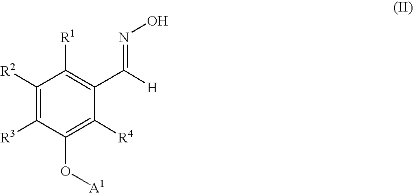

in which
A¹, R¹, R², R³ and R⁴ are as defined above
are reacted with halogenating agents, if appropriate in the presence of one or more diluents,
the substituted benzhydroxamic acid halides, formed in this reaction, of the general formula (III)

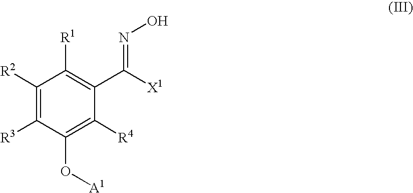

in which
A¹, R¹, R², R³ and R⁴ are as defined above and
X¹ represents halogen
are reacted in situ—i.e. without intermediate isolation—with one or more acid binders
and the arylnitrile N-oxides, formed in this manner, of the general formula (IV)

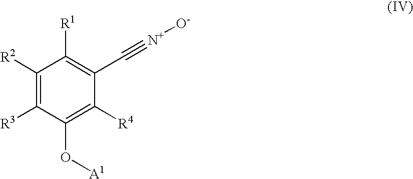

in which
A¹, R¹, R², R³ and R⁴ are as defined above
are reacted in situ—i.e. without intermediate isolation—with alkenes of the general formula (V)

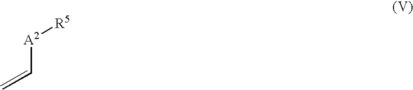

in which
A² and R⁵ are as defined above and
the carbon atoms of the olefinic double bond are optionally substituted as indicated above for Y,
if appropriate in the presence of one or more diluents and if appropriate in the presence of one or more reaction auxiliaries,
and the compounds of the formula (I) obtained in this manner are, if appropriate, converted by customary methods into other compounds of the formula (I).

Using, for example, 2-chloro-5-[(3,3-dichloro-2-propenyl)oxy]benzaldehyde oxime and N-chlorosuccinimide (NCS) in the first reaction step and 5-[4-(allyloxy)phenyl]-2-methyl-2H-tetrazole in the last reaction step as starting materials, the course of the reaction in the process according to the invention can be illustrated by the formula scheme below:

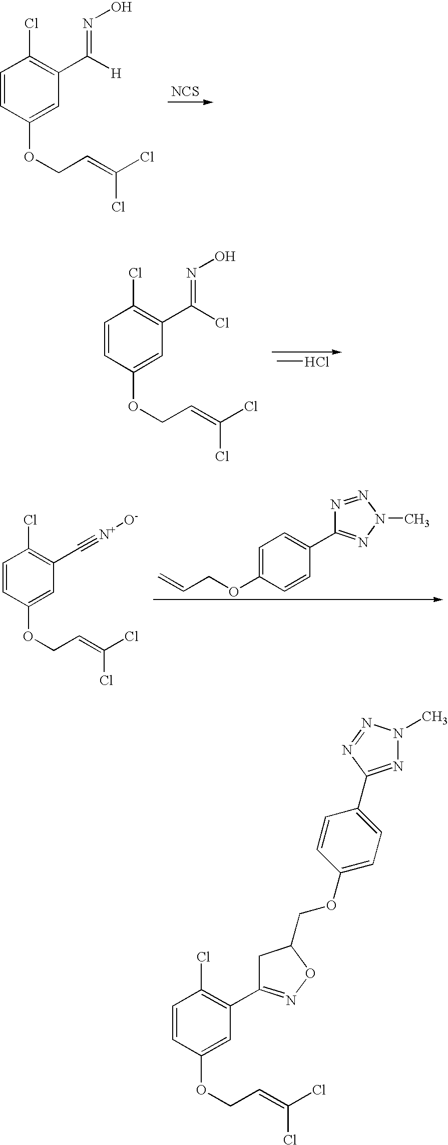

Compounds of the general formula (I) can also be synthesized, for example, as shown schematically below:

(a) by reacting arylnitrile N-oxides of the general formula (IV) with alkynes of the general formula (VI),

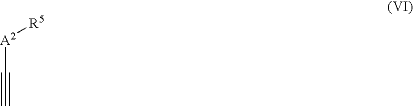

in which
A² and R⁵ are as defined above,
analogously to the description above (cf. also Preparation Examples and also A. R. Kochetkov, S. D. Sokolov: Advances Heterocyclic Chem., A. R. Katritzky, A. J. Boulton (eds.), Vol. 2, New York: Academic Press 1963, p. 365; Houben Weyl, Methoden der Organischen Chemie [Methods of organic chemistry], Volume E8a, pp. 45-176, G. Thieme Verlag, Stuttgart New York), where this reaction may be illustrated as follows:

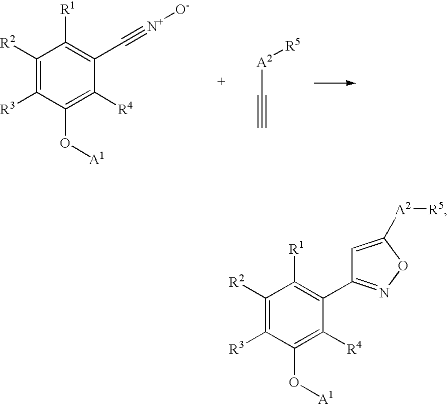

and where R¹, R², R³, R⁴, R⁵, A¹ and A² are as defined above;
(b) by reacting aryl N-oxides of the general formula (IV) with nitrites of the general formula (VII),

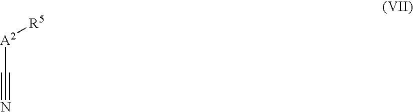

in which
A² and R⁵ are as defined above.
Lit. for 1,2,4-oxadiazoles:
J. H. Boyer: Hereocyclic Compounds, R. C. Elderfield (ed.), Vol. 7, New York, J. Wiley & Sons, 1961, p. 462.
L. B. Clapp. Advandes Heterocyclic Chem., A. R. Katritzky, A. J. Boulton (eds.), Vol. 20, New York, Academic Press 1976, p. 65.

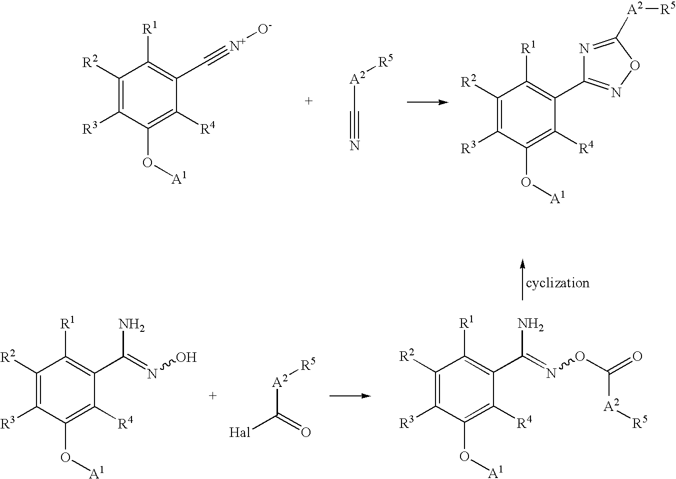

and where R¹, R², R³, R⁴, R⁵, A¹ and A² are as defined above.

Alternatively, it is also possible to prepare the compounds of the formula (I) from corresponding carboxylic acid derivatives, for example an amidoxime and an activated carboxylic acid derivative, for example a carbonyl halide, and subsequent cyclization according to generally known methods, for example,

(α) by reacting carboxylic acid hydrazides with an activated carboxylic acid derivative, for example a carbonyl halide, and subsequent cyclization in the presence of dehydrating agents, for example phosphoryl chloride, according to generally known methods (cf. A. Hetzheim, K. Mockel, In: Advances Heterocyclic Chem., A. R. Katritzky, A. J. Boulton (eds.), Vol. 7, New York: Academic Press 1966, p. 183; J. H. Boyer: Heterocyclic Compounds, R. C. Elderfield (ed.) Vol. 7, New York, J. Wiley & Sons 1961, p. 462), where the reaction may be illustrated as follows:

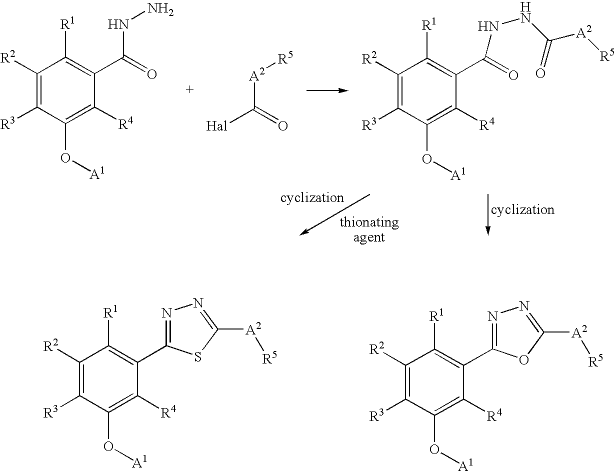

and where R¹, R², R³, R⁴, R⁵, A¹ and A² are as defined above,
and where, if a suitable thionating agent, for example diphosphorus pentasulfide (P₂S₅) or Lawesson's reagent (cf. Review on Lawesson's reagent: R. A. Cherkasov et al., Tetrahedron 41, 1985, p. 2567), is used, the cyclization proceeds in a known manner with incorporation of sulfur (cf. also J. Sandstrom: Advances Heterocyclic Chem., A. R. Katritzky, A. J. Boulton (eds.), Vol. 9, New York: Academic Press 1968, p. 165; L. L. Bambas, five-Membered Heterocyclic Compounds with Nitrogen and Sulfur or Nitrogen, Sulfur, and Oxygen, the Chemistry of Heterocyclic Compounds, Vol. 4, A. Weissberger (ed.), New York, Interscience Publ. 1952, p. 81), or
(β) by reaction of α-haloketo compounds, for example phenacyl halides, with a thioamide according to the generally known Hantzsch method (cf. also R. H. Wiley et al., Org. Reactions 6 (1951) 367; J. M. Sprague, A. H. Land, Heterocyclic Compounds, Elderfield, R. C. (ed.) Vol. 5, New York, J. Wiley & Sons 1957, p. 484), which may be illustrated as follows:

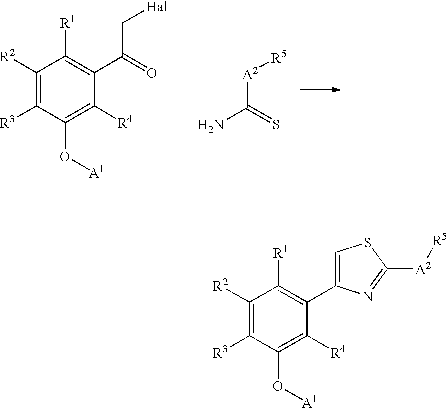

and where R¹, R², R³, R⁴, R⁵, A¹ and A² are as defined above, or
(γ) by reaction of α-haloketo compounds, for example phenacyl halides, with an appropriate amidine according to sufficiently and generally known methods (cf. H. Beyer, Neue Synthesen von Imidazolen und Imidazo-Bicyclen [Novel syntheses of imidazoles and imidazobicycles], Z. Chem. (1970) p. 289; Grimmett, M. R., In: Advances Heterocyclic Chem., A. R. Katritzky, A. J. Boulton (eds.), Vol. 12, New York: Academic Press 1970, p. 104; K. Hoffmann, Imidazole and its Derivatives, The Chemistry of Heterocyclic Compounds, A. Weissberger, Taylor E. C. (eds.), New York, Wiley-Interscience 1953; E. S. Schippper, A. R. Day, Heterocyclic Compounds, R. C. Elderfield (ed.), Vol. 5, New York, J. Wiley & Sons 1957, p. 194), where the reaction may be illustrated as follows:

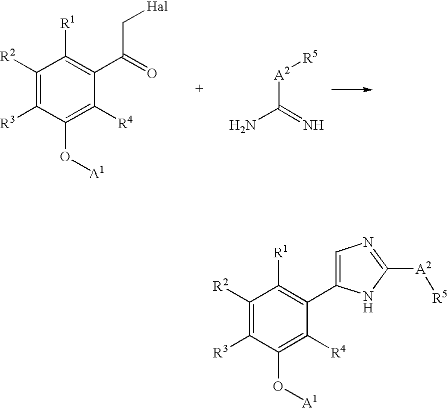

and where R¹, R², R³, R⁴, R⁵, A¹ and A² are as defined above,
(δ) by reaction of activated carboxylic acid derivatives with α-aminoketo compounds to give corresponding acylated α-aminoketo compounds and subsequent and subsequent cyclization in the presence of dehydrating agents, for example phosphorus(V) chloride or thionyl chloride, according to generally known methods (cf. also M. R. Grimmett: Advances Heterocyclic Chem., A. R. Katritzky, A. J. Boulton (eds.), Vol. 12, New York: Academic Press 1970, p. 104; R. J. Ferm, J. L. Riebsommer Chem. Review 54 (1954) p. 593), where the reaction may be illustrated as follows:
(cf. also I. J. Turchi, J. S. Dewar: Chem. Reviews 75, (1975) p. 389; R. Lakhan, B. Ternahi: Advances Heterocyclic Chem., A. R. Katritzky, A. J. Boulton (eds.), Vol. 17, New York: Academic ress 1974, p. 99; J. W. Cornforth: Heterocyclic Compounds, R. C. Elderfield (ed), Vol. New York: Wiley & Sons 1957, p. 298):

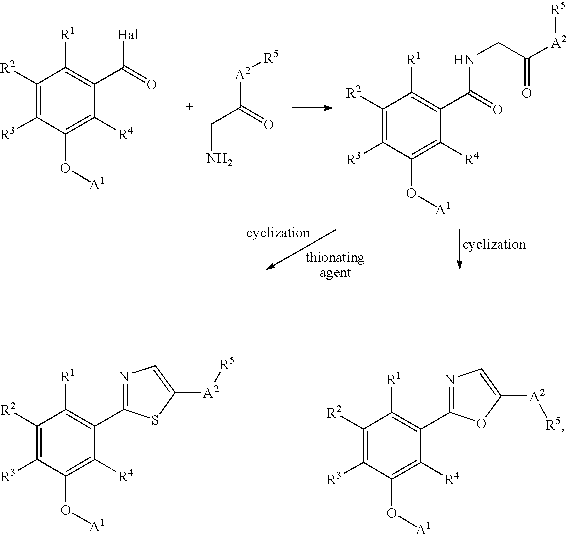

where R¹, R², R³, R⁴, R⁵, A¹ and A² are as defined above,
and where, if a suitable thionating agent, for example diphosphorus pentasulfide (P₂S₅) or Lawesson's reagent (cf. Review on Lawesson's reagent: R. A. Cherkasov et al., Tetrahedron 41, 1985, p. 2567), is used, the cyclization proceeds in a known manner with incorporation of sulfur (cf. also J. M. Sprague, A. H. Land; Heterocyclic Compounds, R. C. Elderfield, Vol. 5, New York, J. Wiley & Sons 1957, p. 484; R. H. Wiley, D. C. England, L. C. Behr, Org. Reactions 6 (1951) 367), or
(ε) by reaction of activated carboxylic acid derivatives with amide hydrazines according to sufficiently and generally known methods (cf. K. T. Potts, Chem. Reviews 61 (1961) 87; J. H. Boyer, Heterocyclic Compounds, R. C. Elderfield (ed.), Vol. 7, New York, J. Wiley & Sons 1961, p. 384), which may be illustrated as follows:

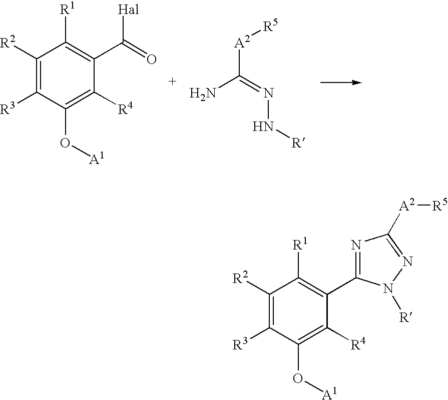

and where R¹, R², R³, R⁴, R⁵, A¹ and A² are as defined above.

The formula (II) provides a general definition of the substituted benzaldoximes to be used as starting materials in the process according to the invention for preparing the compounds of the general formula (I). In the general formula (II), A¹, R¹, R², R³ and R⁴ preferably have those meanings which have already been mentioned above, in connection with the description of the compounds of the general formula (I) according to the invention, as being preferred or as being particularly preferred, very particularly preferred or most preferred for A¹, R¹, R², R³ and R⁴.

The substituted benzaldoximes of the general formula (II) are novel and/or can be prepared by a general process according to BCS 03-3029; the novel compounds are likewise provided by the present application.

The novel substituted benzaldoximes of the general formula (II) are obtained when substituted benzaldehydes of the general formula (VIII)

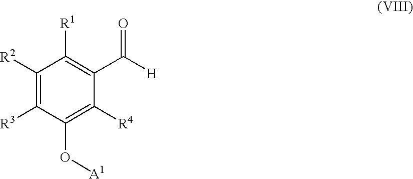

in which
A¹, R¹, R², R³ and R⁴ are as defined above
are reacted with hydroxylamine hydrochloride, if appropriate in the presence of a diluent, such as, for example, acetonitrile or N,N-dimethylformamide, and if appropriate in the presence of a reaction auxiliary, such as, for example, potassium carbonate or triethylamine, at temperatures between 0° C. and 100° C. (cf. Houben-Weyl, Methoden der Organischen Chemie [Methods of organic chemistry], Vol. X/4, 4th ed., 1968, G. Thieme Verlag, Stuttgart New York, p. 55; Vol. 14b, 4th ed. 1990, G. Thieme Verlag, Stuttgart New York, p. 287; J. P. Freemann Chem. Rev. 73 (1973), p. 283.

The halogenation to compounds of the general formula (III) is carried out by initially charging compounds of the general formula (II), if appropriate in a diluent, and adding the appropriate halogenating agent, if appropriate dissolved in a diluent (cf. also Houben-Weyl, Methoder der Organischen Chemie, 4th ed., 1952, G. Thieme Verlag, Stuttgart New York, p. 691; Vol. X/3, 4th ed. 1965, G. Thieme Verlag, Stuttgart-New York, p. 847, Preparation Examples).

The benzaldoximes of the general formula (II) and the compounds of the general formula (III) may, of course, be used in the form of their E or Z isomers and in the form of their mixtures of these stereoisomers.

Some substituted benzaldehydes of the general formula (VIII) (for example compound 3-[(3,3-dichloro-2-propenyl)oxy]benzaldehyde (cf. JP-57018658 and JP-57114503) are already known from the literature (cf. WO 2004/099 197 A2). Novel substituted benzaldehydes of the general formula (VIII) are likewise provided by the present application.

The substituted benzaldehydes of the formula (VIII) are obtained in a manner known per se (cf. Houben-Weyl, Methoden der Organischen Chemie, Volume E3, page 3-608, G. Thieme Verlag, Stuttgart New York), for example by reacting corresponding hydroxybenzoic acid esters of the general formula (IX) with halogen compounds of the general formula (X), followed by hydrolysis of the esters of the general formula (XI), reduction of the carboxylic acids of the general formula (XII) formed in this manner to benzyl alcohols of the general formula (XIII) and oxidation of these compounds, as may be represented, for example, by the reaction scheme below:

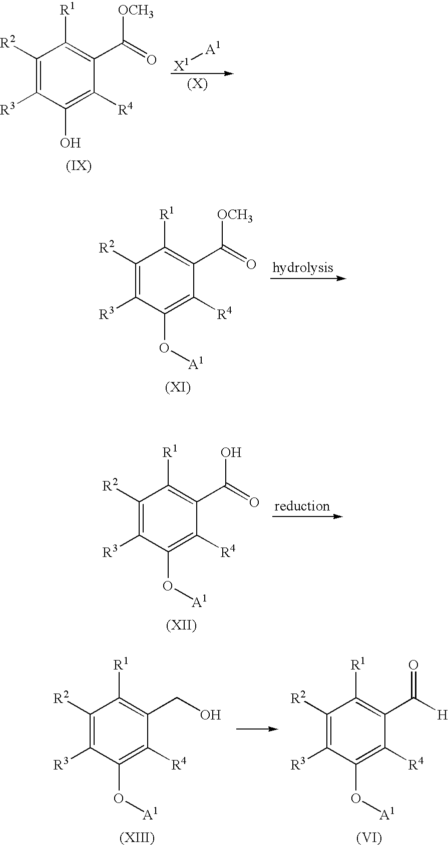

Here, A¹, R¹, R², R³ and R⁴ are as defined above; X¹ represents halogen, in particular chlorine, bromine or iodine.

The precursors of the formulae (XI), (XII) and (XIII) have hitherto not been known from the literature.

If appropriate, the substituents of the compounds of the formula (XIII), such as, for example, the substituent R¹, can also be modified in further reaction steps. In the case that R¹ represents halogen, in particular fluorine, it is possible, for example, to carry out a nucleophilic exchange with suitable nucleophiles in the context of the substituent definition of R¹ in the presence of basic reaction auxiliaries mentioned below (cf., for example, the method from: Bioorg. Med. Chem. 9 (2001) for the N,N-dimethylamino radical, pp. 677-694; J. Med. Chem. 45, 25 (2002) p. 5417, for the isopropylthio radical). Suitable nucleophiles for the exchange reaction are mercapto compounds, hydroxyl compounds or amino compounds.

According to the invention, the aldehydes of the general formula (VIII) can also be prepared by initially generating an aldehyde of the general formula (VIIIb) by generally known methods and then introducing the radical A¹ by means of generally known methods:

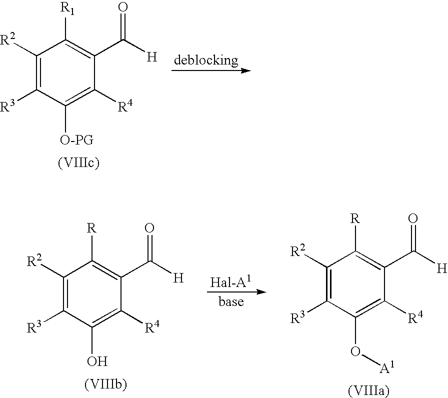

Here, A¹, R¹, R², R³ and R⁴ are as defined above.

In this procedure, it is also possible to use compounds of the general formula (VIIIc) having a suitable protective group (PG) as a precursor for preparing the compounds of the general formula (VIIIc). Examples of protective groups (PG) suitable for hydroxyl groups are substituted methyl ethers and ethers, substituted ethyl ethers, substituted benzyl ethers, silyl ethers, esters, carbonates or sulfonates (cf. Greene T. W., Wuts P. G. W. in Protective Groups in Organic Synthesis; John Wiley & Sons, Inc. 1999).

The formula (V) provides a general definition of the alkenes further to be used as starting materials in the process according to the invention for preparing the compounds of the general formula (I). In the general formula (V), A² and R⁵ preferably have those meanings which have already been mentioned above, in connection with the description of the compounds of the general formula (I) according to the invention, as being preferred or as being particularly preferred, very particularly preferred or most preferred for A² and R⁵.

The starting materials of the general formula (V) are known and/or can be prepared by processes known per se (cf. Preparation Examples).

The first step of the process according to the invention for preparing the compounds of the general formula (I) is carried out using a halogenating agent. Here, suitable halogenating agents are all halogen compounds suitable for converting benzaldehyde oximes into the corresponding benzhydroxamic acid halides. N-bromosuccinimide and N-chlorosuccinimide may be mentioned by way of example.

The process according to the invention for preparing the compounds of the general formula (I) is preferably carried out using one or more acid binders or reaction auxiliaries. Suitable reaction auxiliaries for the process according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate potassium carbonate, cesium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyl-diisopropylamine, N,N-dimethyl-cyclohexylamine, dicyclohexylamine, ethyl-dicyclohexylamine, N,N-dimethyl-aniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN) or 1,8-diazabicyclo[5.4.0]undec-7-ene (DBU). The process according to the invention for preparing the compounds of the general formula (I) is preferably carried out using one or more diluents. Suitable diluents for carrying out the process according to the invention are especially inert organic solvents. These include in particular aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulfoxides, such as dimethyl sulfoxide, alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the process according to the invention, the reaction temperatures can be varied within a relatively wide range. In general, the process is carried out at temperatures between 0° C. and 150° C., preferably between 110° C. and 120° C.

In general, the process according to the invention is carried out under atmospheric pressure. However, it is also possible to carry out the process according to the invention under elevated or reduced pressure—in general between 0.1 bar and 10 bar.

For carrying out the process according to the invention, the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours. Work-up is carried out by customary methods (cf. the Preparation Examples).

By methods known in principle, the compounds of the general formula (I) according to the invention can be converted into other compounds of the general formula (I). Some of these possible conversion reactions are illustrated in an exemplary manner below:

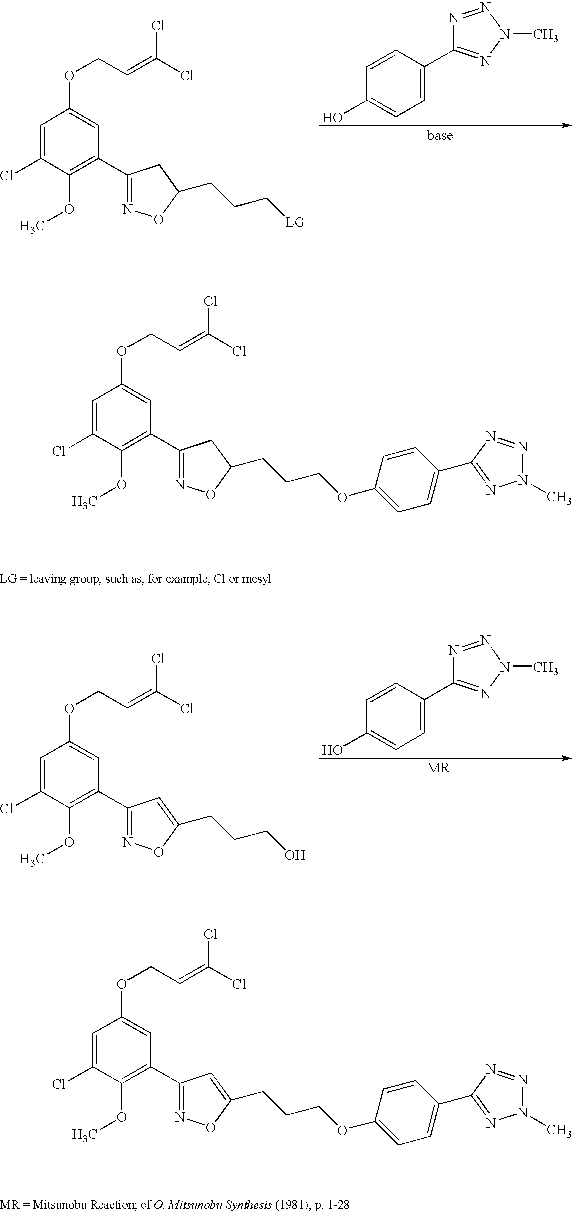

The compounds of the general formula (I) according to the invention are capable of forming salts. Suitable salts of the compounds of the general formula (I) which may be mentioned are customary non-toxic salts, i.e. salts with bases and salts (“adducts”) with acids. Preference is given to salts with inorganic bases, such as alkali metal salts, for example sodium salts, potassium salts or cesium salts, alkaline earth metal salts, for example, calcium salts or magnesium salts, ammonium salts, salts with organic bases, in particular with organic amines, for example triethylammonium salts, dicyclohexylammonium salts, N,N′-dibenzylethylenediammonium salts, pyridinium salts, picolinium salts or ethanolammonium salts, salts with inorganic acids, for example hydrochlorides, hydrobromides, dihydrosulfates, trihydrosulfates, or phosphates, salts with organic carboxylic acids or organic sulfonic acids, for example formates, acetates, trifluoroacetates, maleates, tartrates, methanesulfonates, benzenesulfonates or para-toluenesulfonates.

Salts are formed by standard processes for producing salts. For example, the compounds according to the invention are reacted with appropriate acids to form acid addition salts. Representative acid addition salts are salts which are formed, for example, by reaction with inorganic acids, such as, for example, sulfuric acid, hydrochloric acid, hydrobromic acid, phosphoric acid, or organic carboxylic acids, such as acetic acid, trifluoroacetic acid, citric acid, succinic acid, lactic acid, formic acid, maleic acid, camphoric acid, phthalic acid, glycolic acid, glutaric acid, stearic acid, salicylic acid, sorbic acid, cinnamic acid, picric acid, benzoic acid, or organic sulfonic acids, such as methanesulfonic acid and para-toluenesulfonic acid.

The active compounds are well tolerated by plants, have favorable homeotherm toxicity and are environmentally friendly; they are suitable for protecting plants and plant organs, for increasing harvest yields, for improving the quality of the harvested material and for controlling animal pests, in particular insects, arachnids and nematodes encountered in agriculture, in forests, in gardens and leisure facilities, in the protection of stored products and materials and in the hygiene sector. They can preferably be used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella occidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofinannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

If appropriate, the compounds according to the invention may also be used in certain concentrations or application rates to act as herbicides or as safeners for this purpose, or as microbicides, for example as fungicides, antimycotics or bactericides. If appropriate, they can also be employed as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, habitat or storage space by the customary treatment methods, for example by immersing, spraying, evaporating, fogging, scattering, painting on, injecting and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are:

for example ammonium salts and ground natural minerals such as kaolins, clays, talc, chalk, quartz, attapulgite, montmorillonite or diatomaceous earth, and ground synthetic minerals such as highly disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention may be present in their commercially customary formulations, and in the application forms prepared from these formulations, as mixtures with other active compounds, such as insecticides, attractants, sterilants, bactericides, acaricides, nematicides, fungicides, growth regulators or herbicides. The insecticides include, for example, phosphates, carbamates, carboxylates, chlorinated hydrocarbons, phenylureas, substances produced by microorganisms, etc.

Compounds which are particularly favorable as mixing partners are, for example, the following:

### Fungicides:

2-phenylphenol; 8-hydroxyquinoline sulfate; acibenzolar-5-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4.5]decane-3-amine; sodium tetrathiocarbonate; and copper salts and preparations, such as Bordeaux mixture; copper hydroxide; copper naphthenate; copper oxychloride; copper sulfate; cufraneb; cuprous oxide; mancopper; oxine-copper.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

### Acetylcholine Esterase (AChE) Inhibitors

1.1 Carbamates, for example
   alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb
   Triazamates
1.2 Organophosphates, for example
   acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

### Sodium Channel Modulators/Voltage-Dependent Sodium Channel Blockers

2.1 Pyrethroids, for example
   acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
   DDT
2.2 Oxadiazines, for example indoxacarb

### Acetylcholine Receptor Agonists/Antagonists

3.1 Chloronicotinyls, for example
   acetamiprid, clothianidin, dinotefuran, imidacloprid, nitenpyram, nithiazine, thiacloprid, thiamethoxam
3.2 Nicotine, bensultap, cartap

### Acetylcholine Receptor Modulators

4.1 Spinosyns, for example spinosad

### GABA-Controlled Chloride Channel Antagonists

5.1 Cyclodiene organochlorines, for example
   camphechlor, chlordane, endosulfan, gamma-HCH, HCH, heptachlor, lindane, methoxychlor
5.2 Fiproles, for example
   acetoprole, ethiprole, fipronil, vaniliprole

### Chloride Channel Activators

6.1 Mectins, for example
   avermectin, emamectin, emamectin-benzoate, ivermectin, milbemycin
      Juvenile hormone mimetics, for example
   diofenolan, epofenonane, fenoxycarb, hydroprene, kinoprene, methoprene, pyriproxifen, triprene

### Ecdyson Agonists/Disruptors

8.1 Diacylhydrazines, for example
   chromafenozide, halofenozide, methoxyfenozide, tebufenozide

### Chitin Biosynthesis Inhibitors

9.1 Benzoylureas, for example
   bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
9.2 Buprofezin
9.3 Cyromazine

### Oxidative Phosphorylation Inhibitors, ATP Disruptors

10.1 Diafenthiuron
10.2 Organotins, for example azocyclotin, cyhexatin, fenbutatin-oxide

### Oxidative Phosphorylation Decouplers Acting by Interrupting the H-Proton Gradient

11.1 Pyrroles, for example chlorfenapyr
11.2 Dinitrophenols, for example binapacyrl, dinobuton, dinocap, DNOC

### Side-I Electron Transport Inhibitors

12.1 METIs, for example fenazaquin, fenpyroximate, pyrimidifen, pyridaben, tebufenpyrad, tolfenpyrad
12.2 Hydramethylnon
12.3 Dicofol

### Side-II Electron Transport Inhibitors

Rotenone

### Side-III Electron Transport Inhibitors

Acequinocyl, fluacrypyrim

### Microbial Disruptors of the Insect Gut Membrane

Bacillus thuringiensis strains

### Fat Synthesis Inhibitors

Tetronic acids, for example
   spirodiclofen, spiromesifen
Tetramic acids, for example
   3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS-Reg.-No.: 382608-10-8) and carbonic acid, cis-3-(2,5-dimethylphenyl)-8-methoxy-2-oxo-1-azaspiro[4.5]dec-3-en-4-yl ethyl ester (CAS-Reg.-No.: 203313-25-1)
      Carboxamides, for example flonicamid
      Octopaminergic agonists, for example amitraz
      Inhibitors of magnesium-stimulated ATPase, for example propargite
      BDCAs, for example N2-[1,1-dimethyl-2-(methylsulfonyl)ethyl]-3-iodo-N1-[2-methyl-4-[1,2,2,2-tetrafluoro-1-(trifluoromethyl)ethyl]phenyl]-1,2-benzenedicarboxamide (CAS-Reg.-No.: 272451-65-7)
      Nereistoxin analogs, for example thiocyclam hydrogen oxalate, thiosultap-sodium
      Biologicals, hormones or pheromones, for example
azadirachtin, Bacillus spec., Beauveria spec., codlemone, Metarrhizium spec., Paecilomyces spec., Thuringiensin, Verticillium spec.
   Active Compounds with Unknown or Unspecific Mechanisms of Action
23.1 Fumigants, for example aluminum phosphide, methyl bromide, sulfuryl fluoride
23.2 Selective antifeedants, for example
   cryolite, flonicamid, pymetrozine
23.3 Mite growth inhibitors, for example
   clofentezine, etoxazole, hexythiazox
23.4 Amidoflumet, benclothiaz, benzoximate, bifenazate, bromopropylate, buprofezin, chinomethionat, chlordimeform, chlorobenzilate, chloropicrin, clothiazoben, cycloprene, cyflumetafen, dicyclanil, fenoxacrim, fentrifanil, flubendiamide, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,
   and also products which comprise insecticidal plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001 up to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants and their parts in accordance with the invention. In a preferred embodiment, wild plant species or plant varieties and plant cultivars which have been obtained by conventional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetic modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects.

Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processability of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or better processability of the harvested products. Further examples of such traits, which must be mentioned especially, are better defense of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soybeans, potato, cotton, tobacco, oilseed rape and fruit plants (with the fruits apples, pears, citrus fruits and grapes), with particular emphasis on maize, soybeans, potatoes, cotton, tobacco, and oilseed rape. Traits which are especially emphasized are the increased defense of the plants against insects, arachnids, nematodes and slugs and snails, owing to toxins being formed in the plants, in particular toxins which are generated in the plants by the genetic material of Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations; hereinbelow “Bt plants”). Other traits which are particularly emphasized are the increased defense of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulfonylureas, glyphosate or phosphinotricin (for example “PAT” gene). The genes which confer the desired traits in each case may also be present in the transgenic plants in combination with one another. Examples of “Bt plants” which may be mentioned are maize cultivars, cotton cultivars, soybean cultivars and potato cultivars which are commercially available under the trade names YIELD GARD® (for example maize, cotton, soybeans), KnockOut® (for example maize), StarLink® (for example maize), Boligard® (cotton), Nucotn® (cotton) and NewLeaf® (potato). Examples of herbicide-tolerant plants which may be mentioned are maize cultivars, cotton cultivars and soybean cultivars which are commercially available under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned also include the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously according to the invention with the compounds of the general formula I or the active compound mixtures according to the invention. The preferred ranges stated above for the active compounds and mixtures also apply to the treatment of these plants.

Particular emphasis may be given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and licking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Wemeckiella spp., Lepikentron spp., Damalina spp., Trichodectes spp., Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as Hylotrupes bajulus, Chlorophorus pilosis, Anobium punctatum, Xestobium rufovillosum, Ptilinus pecticomis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Dermapterans, such as Sirex juvencus, Urocerus gigas, Urocerus gigas taignus, Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:

construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of terpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a flashpoint of above 30° C., preferably above 45° C., and that the insecticide/fungicide mixture is soluble or emulsifiable in this solvent mixture.

In a preferred embodiment, part of the organochemical solvent or solvent mixture or an aliphatic polar organochemical solvent or solvent mixture is replaced. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/coumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odor-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di(2-ethylhexyl) adipate, the stearates, such as butyl stearate or amyl stearate, the oleates, such as butyl oleate, the glycerol ethers or relatively high-molecular-weight glycol ethers, glycerol esters and p-toluenesulfonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide, triflumuron, chlothianidin, spinosad, tefluthrin, and fungicides, such as epoxyconazole, hexaconazole, azaconazole, propiconazole, tebuconazole, cyproconazole, metconazole, imazalil, dichlorfluanid, tolylfluanid, 3-iodo-2-propynylbutyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signaling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action. Using the compounds according to the invention, alone or in combination with other active compounds, allows the use of heavy metals such as, for example, in bis(trialkyltin) sulfides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulfide, antimony oxide, polymeric butyl titanate, phenyl-(bispyridine)-bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;
molluscicides such as fentin acetate, metaldehyde, methiocarb, niclosamid, thiodicarb and trimethacarb, Fe chelates;
or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium, copper, sodium and zinc salts of 2-pyridinethiol 1-oxide, pyridine/triphenylborane, tetrabutyldistannoxane, 2,3,5,6-tetrachloro-4-(methylsulfonyl)-pyridine, 2,4,5,6-tetrachloroisophthalonitrile, tetramethylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Example (I-1)

### (R/S)-3-(3-Chloro-2-methoxy-5-(1,1-dichloro-1-propen-3-oxy)phenyl)-5-((4-(N-2-ethyltetrazol-5-yl))phenyl)-3-(propyl)ether-1-yl)-Δ²-isoxazoline

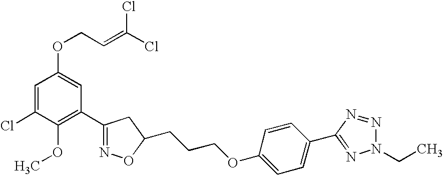

### Step 1

### Preparation of (R/S)-3-(5-benzyloxy-3-chloro-2-methoxyphenyl)-5-(3-hydroxypropyl)-Δ²-isoxazoline

1.45 g (5.0 mmol) of 3-chloro-5-benzyloxy-2-methoxybenzaldehyde oxime are dissolved in 100 ml of N,N-dimethylformamide (DMF), and 0.73 (5.5 mmol) of N-chlorosuccinimide (NCS) is added. The clear yellowish reaction mixture is then stirred at room temperature for about 24 hours. 6.45 g (7.5 mmol) of 4-penten-1-ol and 5.56 g (5.5 mmol) of triethylamine are then added, and the yellowish solution is stirred at room temperature for about 24 hours. For work-up, the reaction solution is added to about 200 ml of water, and the milky solution is extracted with dichloromethane. The organic phase is separated off and then dried over sodium sulfate and filtered off, and the filtrate is concentrated under reduced pressure.

This gives 1.85 g (98% of theory) of (R/S)-3-(5-benzyloxy-3-chloro-2-methoxyphenyl)-5-(3-hydroxypropyl)-Δ²-isoxazoline as a yellow-brown viscous product which can be used for subsequent reactions.

LC-MS: 376 (M⁺)

### Step 2

### Preparation of (R/S)-3-(5-benzyloxy-3-chloro-2-methoxyphenyl)-5-((4-(N-2-ethyltetrazol-5-yl))phenyl)-3-(propyl)ether-1-yl)-Δ²-isoxazoline

300.0 mg (0.65 mmol) of (R/S)-3-(5-benzyloxy-3-chloro-2-methoxyphenyl)-5-(3-hydroxypropyl)-Δ²-isoxazoline and 124.5 mg (0.65 mmol) of 4-(2-N-ethyltetrazol-5-yl)phenol are stirred in 20 ml of tetrahydrofuran (THF), and 343.3 mg of diethyl azodicarboxylate (DEAD), dissolved in 2 ml of THF, are added (cf. O. Mitsunobu, Synthesis 1-28, 1981). The reaction mixture is then stirred at room temperature for about 18 hours, and the reaction is monitored by thin-layer chromatography (TLC) (cyclohexane:acetone=2:1). The turbid reaction solution is concentrated under reduced pressure, the residue that remains is taken up in ethyl acetate and washed with water. The separated organic phase is dried over magnesium sulfate and filtered, and the filtrate is concentrated under reduced pressure. The residue that remains (contains 21% of target product according to LC-MS) is purified by HPLC (gradient: standard/neutral) to a purity of >97%.

This gives 113 mg (31% of theory) of (R/S)-3-(5-benzyloxy-3-chloro-2-methoxyphenyl)-5-((4-(N-2-ethyltetrazol-5-yl))phenyl)-3-(propyl)ether-1-yl)-Δ²-isoxazoline.

LC-MS: 548 (M⁺)

### Step 3

### Preparation of (R/S)-3-(3-chloro-5-hydroxy-2-methoxyphenyl)-5-((4-(N-2-ethyltetrazol-5-yl))phenyl)-3-(propyl)ether-1-yl)-Δ²-isoxazoline

110 mg (0.20 mmol) of (R/S)-3-(5-benzyloxy-3-chloro-2-methoxyphenyl)-5-((4-(N-2-ethyltetrazol-5-yl))phenyl)-3-(propyl)ether-1-yl)-Δ²-isoxazoline in 20 ml of ethanol are hydrogenated in the presence of 22 mg of Pd(OH)₂/carbon [Pd content 20%] until the hydrogen uptake has ended. The catalyst is filtered off, and the entire reaction solution is then concentrated under reduced pressure.

This gives 84 mg (73% of theory) of (R/S)-3-(3-chloro-5-hydroxy-2-methoxyphenyl)-5-((4-(N-2-ethyltetrazol-5-yl))phenyl)-3-(propyl)ether-1-yl)-Δ²-isoxazoline which can be alkylated in the next reaction step.

LC-MS: 458 (M⁺)

### Step 4

### Preparation of (R/S)-3-(3-chloro-2-methoxy-5-(1,1-dichloro-1-propen-3-oxy)phenyl)-5-((4-(N-2-ethyltetrazol-5-yl))phenyl)-3-(propyl)ether-1-yl)-Δ²-isoxazoline

Under nitrogen as protective gas, 80 mg (0.17 mmol) of (R/S)-3-(3-chloro-5-hydroxy-2-methoxyphenyl)-5-((4-(N-2-ethyltetrazol-5-yl))phenyl)-3-(propyl)ether-1-yl)-Δ²-isoxazoline are initially charged in 3.0 ml of dry DMF, and 57.3 mg (0.17 mmol) of cesium carbonate and 33.4 mg (0.17 mmol) of 3-bromo-1,1-dichloropropene (97% pure) are then added with stirring. The reaction mixture is stirred at room temperature for another 4 days approximately and then concentrated under reduced pressure. The residue that remains (contains 86% of target product according to LC-MS) is purified by HPLC.

This gives 50 mg (75% of theory) of (R/S)-3-(3-chloro-2-methoxy-5-(1,1-dichloro-1-propen-3-oxy)phenyl)-5-((4-(N-2-ethyltetrazol-5-yl))phenyl)-3-(propyl)ether-1-yl)-Δ²-isoxazoline.

LC-MS: 566 (M⁺)

### Example (I-2)

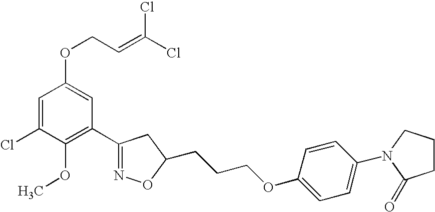

(R/S)-3-(3-Chloro-2-methoxy-5-(1,1-dichloro-1-propen-3-oxy)phenyl)-5-((4-(N-2-oxopyrrolidino))phenyl)-3-(propyl)ether-1-yl)-Δ²-isoxazoline is prepared analogously to the reaction procedure of example (1-2).

### Step 2

The Mitsunobu reaction is carried out using:

| | |
|---|---|
| 300.0 mg (0.65 mmol) of | (R/S)-3-(3-chloro-5-benzyloxy-2- |
| | methoxyphenyl)-5-hydroxyethyl-Δ²-isoxazoline |
| 115.9 mg (0.65 mmol) of | 4-(N-2-oxopyrrolidino)phenol |
| 188.8 mg (0.72 mmol) of | triphenylphosphine |
| 125.3 mg (0.72 mmol) of | diethyl azodicarboxylate (DEAD) |
| 22 ml of | tetrahydrofuran |

The crude product that remains is purified by preparative HPLC (gradient: standard/neutral) to a purity of >96%. This gives 133 mg (36.5% of theory) of (R/S)-3-(2-chloro-2-methoxy-5-benzyloxyphenyl)-5-((4-(N-2-oxopyrrolidino))phenyl)-3-(propyl)ether-1-yl)-Δ²-isoxazoline.

| | |
|---|---|
| LC-MS (ESI positive) m/e = 535 (M⁺) | C₃₀H₃₁ClN₂O₅ (535.0) |

### Step 3

The O-benzyl radical is removed by hydrogenation using:

| | |
|---|---|
| 136.0 mg (0.25 mmol) of | (R/S)-3-(2-chloro-2-methoxy-5- |
| | benzyloxyphenyl)-5-((4-(N-2-oxopyrrolidino))- |
| | phenyl)-3-(propyl) ether-1-yl)-Δ²- |
| | isoxazoline |
| 26.4 mg (0.18 mmol) of | palladium(II) hydroxide/carbon [Pd content |
| | 20%] |
| 20 ml of | ethanol |

The crude product that remains is purified by preparative HPLC (gradient: standard/neutral) to a purity of >94%. This gives 43 mg (35.3% of theory) of (R/S)-3-(2-chloro-5-hydroxy-2-methoxy-5-phenyl)-5-((4-(N-2-oxopyrrolidino))phenyl)-3-(propyl)ether-1-yl)-Δ²-isoxazoline.

| | |
|---|---|
| LC-MS (ESI positive) m/e = 445 (MH⁺) | C₂₃H₂₅ClN₂O₅ (444.9) |

### Step 4

The O-alkylation is carried out using:

| | |
|---|---|
| 40.0 mg (0.08 mmol) of | (R/S)-3-(2-chloro-5-hydroxy-2-methoxy-5- |
| | phenyl)-5-((4-(N-2-oxopyrrolidino))phenyl)-3- |
| | (propyl) ether-1-yl)-Δ²-isoxazoline |
| 23.8 mg (0.12 mmol) of | 3-bromo-1,1-dichloropropene |
| 40.8 mg (0.12 mmol) of | cesium carbonate |
| 3 ml of | N,N-dimethylformamide |

The crude product that remains is purified by preparative HPLC (gradient: standard/neutral) to a purity of >99%. This gives 32 mg (69% of theory) of (R/S)-3-(3-chloro-2-methoxy-5-(1,1-dichloro-1-propen-3-oxy)phenyl)-5-((4-(N-2-oxopyrrolidinophenyl))-3-(propyl)ether-1-yl)-Δ²-isoxazoline.

| | |
|---|---|
| LC-MS (ESI positive) m/e = 553 (M⁺) | C₂₆H₂₇Cl₃N₂O₅ (553.8) |

### Example (I-3)

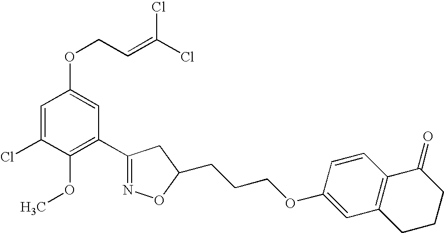

(R/S)-3-(3-Chloro-2-methoxy-5-(1,1-dichloro-1-propen-3-oxy)phenyl)-5-((1-tetralon-6-yl)-3-(propyl)ether-1-yl)-Δ²-isoxazoline is prepared analogously to the reaction procedure of example (1-2), step 1.

The [3+2]-cyclization reaction is carried out using:

| | |
|---|---|
| 122.0 mg (0.39 mmol) of | 3-chloro-5-benzyloxy-2-methoxybenzaldehyde |
| | oxime |
| 108.5 mg (0.47 mmol) of | 6-(4-penten-1-yloxy)-1-tetralone |
| 62.9 mg (0.47 mmol) of | N-chlorosuccinimide |
| 59.6 mg (0.58 mmol) of | triethylamine |
| 50 ml of | N,N-dimethylformamide |

The crude product that remains is purified on a silica gel column (silica gel 60—Merck, particle size: 0.04 to 0.063 mm) using the mobile phase cyclohexane:acetone (20:1). This gives 92 mg (38.5% of theory) of (R/S)-3-(3-chloro-2-methoxy-5-(1,1-dichloro-1-propen-3-oxy)phenyl)-5-((1-tetralon-6-yl)-3-(propyl)ether-1-yl)-Δ²-isoxazoline.

| | |
|---|---|
| LC-MS (ESI positive) m/e = 538 (M⁺) | C₂₆H₂₈Cl₃NO₅ (538.8) |

¹H-NMR (CD₃CN, 400 MHz) δ=2.08 (2H, —CH₂-tetralone); 2.52 (2H, —CH₂-tetralone); 2.91 (2H, —CO—CH₂-tetralone); 3.10 and 3.49 (in each case 1H, diastereotopic —N═CH₂—, isoxazoline); 3.76 (3H, —O—CH₃); 4.12 (2H, —CH₂—O-arom.); 4.66 (2H, —CH₂—CH═CCl₂); 4.77 (1H, —CH—O—, isoxazoline); 6.24 (1H, CH═CCl₂); 6.79-6.85 (2H, arom.); 7.09-7.11 (2H, arom.); 7.87 (1H, tetralone) ppm.

### Example (I-4)

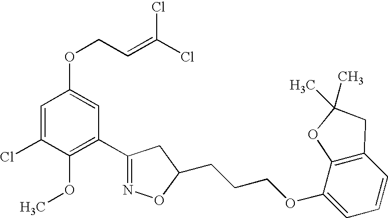

(R/S)-3-(3-Chloro-2-methoxy-5-(1,1-dichloro-1-propen-3-oxy)phenyl)-5-((2,3-dihydro-2,2-dimethylbenzofuran-7-yl)-3-(propyl)ether-1-yl)-Δ²-isoxazoline is prepared analogously to the reaction procedure of example (1-2), step 1.

The [3+2]-cyclization reaction is carried out using:

| | |
|---|---|
| 200.0 mg (0.64 mmol) of | 3-chloro-5-benzyloxy-2-methoxybenzaldehyde |
| | oxime |
| 179.5 mg (0.77 mmol) of | (2,3-dihydro-2,2-dimethylbenzofuran- |
| | 7-yl) 3-(4-penten-1-yl) ether |
| 103.1 mg (0.77 mmol) of | N-chlorosuccinimide |
| 97.7 mg (0.96 mmol) of | triethylamine |
| 20 ml of | N,N-dimethylformamide |

The crude product that remains is purified on a silica gel column (silica gel 60—Merck, particle size: 0.04 to 0.063 mm) using the mobile phase cyclohexane:acetone (20:1). This gives 104 mg (27.4% of theory) of (R/S)-3-(3-chloro-2-methoxy-5-(1,1-dichloro-1-propen-3-oxy)phenyl)-5-((2,3-dihydro-2,2-dimethylbenzofuran-7-yl)-3-(propyl)ether-1-yl)-Δ²-isoxazoline.

| | |
|---|---|
| LC-MS (ESI positive) m/e = 540 (MH⁺) | C₂₆H₂₈Cl₃NO₅ (540.8) |

¹H-NMR (CD₃CN, 400 MHz) δ=3.00 (2H, —CH₂-arom); 3.10 and 3.49 (in each case 1H, diastereotopic —N═CH₂—, isoxazoline); 3.76 (3H, —O—CH₃); 4.07 (2H, —CH₂—O-arom.); 4.66 (2H, —CH₂—CH═CCl₂); 4.77 (1H, —CH—O—, isoxazoline); 6.24 (1H, CH═CCl₂); 6.70-6.78 (3H, arom.); 7.09 (2H, arom.) ppm.

### Example (I-5)

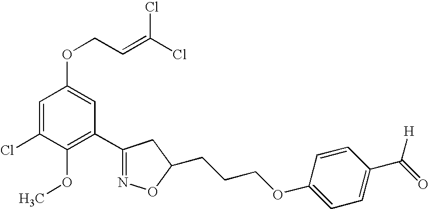

In a first reaction step, (R/S)-3-(3-chloro-2-methoxy-5-(1,1-dichloro-1-propen-3-oxy)phenyl)-5-(4-formylphenyl)-3-(propyl)ether-1-yl)-Δ²-isoxazoline is prepared analogously to the reaction procedure of example (1-2), step 1.

The [3+2]-cyclization reaction is carried out using:

| | |
|---|---|
| 408.1 mg (1.31 mmol) of | 3-chloro-5-benzyloxy-2-methoxybenzaldehyde |
| | oxime |
| 300.0 mg (1.57 mmol) of | (O-pent-4-en-1-yl)-4-hydroxybenzaldehyde |
| 210.5 mg (1.57 mmol) of | N-chlorosuccinimide |
| 199.4 mg (1.97 mmol) of | triethylamine |
| 40 ml of | N,N-dimethylformamide |

The crude product that remains is purified on a silica gel column (silica gel 60—Merck, particle size: 0.04 to 0.063 mm) using the mobile phase cyclohexane:acetone (4:1). This gives 313 mg (45.3% of theory) of (R/S)-3-(3-chloro-2-methoxy-5-(1,1-dichloro-1-propen-3-oxy)phenyl)-5-(4-formylphenyl)-3-(propyl)ether-1-yl)-Δ²-isoxazoline.

| | |
|---|---|
| LC-MS (ESI positive) m/e = 498 (M⁺) | C₂₃H₂₂Cl₃NO₅ (498.8) |

¹H-NMR (CD₃CN, 400 MHz) δ=3.10 and 3.49 (in each case 1H, diastereotopic —N═CH₂—, isoxazoline); 3.76 (3H, —O—CH₃); 4.16 (2H, —CH₂—O-arom.); 4.66 (2H, —CH₂—CH═CCl₂); 4.78 (1H, —CH—O—, isoxazoline); 6.24 (1H, CH═CCl₂); 7.04-7.11 (4H, arom.); 7.82 (2H, arom.); 9.89 (s, 1H, —CHO) ppm.

### Example (I-6)

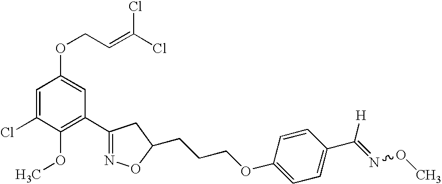

(R/S)-3-(3-Chloro-2-methoxy-5-(1,1-dichloro-1-propen-3-oxy)phenyl)-5-(4-methoxyiminophenyl)-3-(propyl)ether-1-yl)-Δ²-isoxazoline is prepared from (R/S)-3-(3-chloro-2-methoxy-5-(1,1-dichloro-1-propen-3-oxy)phenyl)-5-(4-formylphenyl)-3-(propyl)ether-1-yl)-Δ²-isoxazoline:

250 mg (0.5 mmol) of (R/S)-3-(3-chloro-2-methoxy-5-(1,1-dichloro-1-propen-3-oxy)phenyl)-5-(4-formylphenyl)-3-(propyl)ether-1-yl)-Δ²-isoxazoline are stirred in a mixture of 5 ml of water and 10 ml of ethanol (ratio 1:2), and 46.0 mg (0.5 mmol) of O-methylhydroxylamine hydrochloride and 45.2 mg (0.55 mmol) of sodium acetate are added. The reaction mixture is then stirred at 50° C. for one hour and at room temperature for a further 18 hours. The entire reaction mixture is then concentrated under reduced pressure and the aqueous solution that remains is extracted three times with ethyl acetate. This gives 254 mg (45.3% of theory) of (R/S)-3-(3-chloro-2-methoxy-5-(1,1-dichloro-1-propen-3-oxy)phenyl)-5-(4-methoxyiminophenyl)-3-(propyl)ether-1-yl)-Δ²-isoxazoline as an (E/Z) isomer mixture.

| | |
|---|---|
| LC-MS (ESI positive) m/e = 527 (M⁺) | C₂₄H₂₅Cl₃N₂O₅ (527.8) |

¹H-NMR (CD₃CN, 400 MHz) δ=3.11 and 3.50 (in each case 1H, diastereotopic —N═CH₂—, isoxazoline); 3.76 (3H, —O—CH₃); 3.87 (3H, ═N—O—CH₃); 4.08 (2H, —CH₂—O-arom.); 4.66 (2H, —CH₂—CH═CCl₂); 4.78 (1H, —CH—O—, isoxazoline); 6.24 (1H, CH═CCl₂); 6.92 (2H, arom); 7.10 (2H, arom.); 7.50 (2H, arom.); 8.03 (s, 1H, —CHN—O—) ppm.

### Starting Materials of the Formula (II):

### Example (II-1)

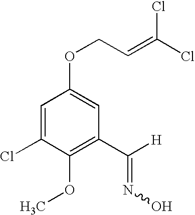

### Step 1

### Preparation of methyl 3-chloro-2,5-dihydroxybenzoate

Under nitrogen as protective gas, 6.75 g (40 mmol) of methyl 2,5-dihydroxybenzoate are dissolved in 80 ml of dry DMF. With stirring, a total of 6.94 (5.2 mmol) of N-chlorosuccinimide (NCS) are added at room temperature a little at a time (i.e. about 1 g every 30 minutes), the color of the reaction solution slowly changing to red. After the addition has ended, the mixture is stirred overnight (at RT) to allow the reaction to go to completion. The progress of the chlorination can be monitored by thin-layer chromatography (mobile phase n-hexane/ethyl acetate 1:1) by assessing the reduction of the starting material spot; the mixture is worked up only once hardly any starting material can be detected by thin-layer chromatography—if required, more NCS is added. For work-up, the mixture is shaken in a separating funnel with 200 ml of water and extracted with a mixture of 200 ml of heptane and 200 ml of ethyl acetate. The organic phase is washed once more with about 100 to 200 ml of water, and the solvent is then removed under reduced pressure (rotary evaporator bath temperature increased to about 70° C./15 mbar to remove remaining DMF). What remains is a brown solid (about 9 g) (if what is separated off is not a solid but an oil, this has to be taken up once more in n-hexane/ethyl acetate (1:1) and washed with water), which is recrystallized from 200 ml of n-heptane in the presence of about 10 ml of ethyl acetate (rotary evaporator bath temperature 85° C., triturated/crystallized at room temperature), giving, after filtration with suction and drying, initially 2.4 g of flesh-colored crystals. From the mother liquor, a further 2.4 g of product can be obtained from a further crystallization (concentration of the mother liquor to dryness, recrystallization of the residue).

This gives 4.8 g (59% of theory) of methyl 3-chloro-2,5-dihydroxybenzoate.

Melting point: 126° C.

MS (ES−): 201

¹H-NMR (300 MHz, CDCl₃): δ (ppm)=3.96 (s, 3H); 4.61 (s, 1H); 7.15 (d, 1H); 7.24 (d, 1H); 10.86 (s, 1H).

### Step 2

### Preparation of methyl 3-chloro-2,5-bis(3,3-dichloroallyloxy)dihydroxybenzoate

Under nitrogen as protective gas, 5.4 g (177 mmol) of sodium hydride (80% pure) are initially charged in about 200 ml of dry DMF, and a solution of 16.3 g (80.4 mmol) of methyl 3-chloro-2,5-dihydroxybenzoate (dissolved in about 40 ml of dry DMF) is then added dropwise with stirring. Hydrogen evolves, and the color of the solution changes to red-brown; during the addition, the contents of the flask are kept at a temperature of 25-30° C. using a water bath. Once the evolution of hydrogen has ended, the mixture is stirred vigorously at room temperature for another 20 minutes, 34 g (173 mmol) of 3-bromo-1,1-dichloropropene (97% pure) are then added dropwise over a period of about 30 minutes and the mixture is stirred for another 1 to 2 hours. For work-up, about 400 ml of water are added, the mixture is extracted with methylene chloride (2×250 ml), and the combined organic phases are washed once with water and concentrated to dryness. What remains is a brown oil which is purified by column chromatography on silica gel (the column is conditioned with n-hexane/ethyl acetate (9:1); elution with 9:1, with increasing polarity to 1:1). The desired product elutes first. Concentration of the appropriate fractions gives a yellow oil which crystallizes after prolonged standing to afford a pale yellow solid.

This gives 23.9 g (71% of theory) of methyl 3-chloro-2,5-bis(3,3-dichloroallyloxy)benzoate.

Melting point: 63° C.

¹H-NMR (300 MHz, CDCl₃): δ (ppm)=3.92 (s, 3H); 4.65 (m, 1H); 6.12 (t, 1H); 6.32 (t, 1H); 7.12 (d, 1H); 7.23 (d, 1H).

### Step 3

### Preparation of methyl 3-chloro-5-(3,3-dichloroallyloxy)-2-hydroxybenzoate

Under nitrogen as protective gas, 9.8 g (38.0 mmol) of powdered magnesium bromide etherate are suspended in 200 ml of toluene and heated with vigorous stirring to about 120° C. With stirring, a solution of 10 g (23.8 mmol) of methyl 3-chloro-2,5-bis(3,3-dichloroallyloxy)benzoate in about 50 ml of toluene is added dropwise to this hot suspension, and the mixture is stirred at about 120° C. for another 2-4 hours. The progress of the reaction can be analyzed by TLC by monitoring the disappearance of the starting material spot. Once no more starting material is detected, the mixture is allowed to cool to room temperature and then poured into about 50 ml of conc. hydrochloric acid in a separating funnel, the phases are mixed and another 100 ml of water (approximately) are added. The organic phase is separated off, the aqueous phase is extracted two more times with about 200 ml of toluene and the combined organic phases are concentrated to dryness. This also results in 3-bromo-1,1-dichloropropene, which is formed in the reaction and has a slightly pungent and irritating smell, distilling over. The residue is recrystallized from methanol (60° C./RT) and filtered off with suction, and the colorless crystals are dried on a frit. If appropriate, further product can be isolated from the mother liquor by a second crystallization.

This gives 5.56 g (75% of theory).

Melting point: 86° C.

MS (ES+): 311

¹H-NMR (300 MHz, CDCl₃): δ (ppm)=3.98 (s, 3H); 4.60 (d, 2H); 6.12 (t, 1H); 7.20 (d, 1H); 7.27 (d, 1H); 10.94 (s, 1H).

### Step 4

### Preparation of methyl 3-chloro-5-(3,3-dichloroallyloxy)-2-methoxybenzoate

Under nitrogen as protective gas, 7.0 g (22.5 mmol) of methyl 3-chloro-5-(3,3-dichloroallyloxy)-2-hydroxybenzoate and 12.8 g (102 mmol) of dimethyl sulfate are initially charged at room temperature in 150 ml of dry DMF, and 13.2 g (95.2 mmol) of dry potassium carbonate are added with vigorous stirring. Initially, a light-yellow suspension is obtained, and after a number of minutes, the reaction becomes exothermic, resulting in the color of the suspension changing to dark yellow. The mixture is stirred at room temperature for another 2 hours approximately, and the suspension is then poured into about 300 ml of water and extracted twice with 400 ml of dichloromethane. Concentration of the combined organic phases to dryness affords an oily residue.

This gives 7.05 (96% of theory) of methyl 3-chloro-5-(3,3-dichloroallyloxy)-2-methoxybenzoate.

¹H-NMR (300 MHz, CDCl₃): δ (ppm)=3.89 (s, 3H); 3.95 (s, 3H); 4.63 (d, 2H); 6.12 (t, 1H); 7.10 (d, 1H); 7.21 (d, 1H).

### Step 5

### Preparation of 3-chloro-5-(3,3-dichloroallyloxy)-2-methoxybenzoic acid

8.0 g (24.6 mmol) of methyl 3-chloro-5-(3,3-dichloroallyloxy)-2-methoxybenzoate are dissolved in about 100 ml of methanol, and about 40 ml of 10% strength aqueous sodium hydroxide solution are added. On a rotary evaporator, the emulsion is heated to about 50° C. (“with stirring”), and a pale yellow solution is formed. From time to time, the progress of the hydrolysis is checked by TLC; once no more starting material can be detected by TLC (generally after 20 minutes), most of the methanol is distilled off, and the aqueous solution is transferred into an Erlenmeyer flask and cooled in an ice-bath. With vigorous stirring, concentrated hydrochloric acid is then added until the reaction is clearly acidic, resulting in the product separating off as a colorless solid. The precipitate is filtered off with suction, taken up in methylene chloride and washed with water to remove salts that have been carried along, and the organic phase is concentrated to dryness. What remains is a colorless solid.

This gives 6.8 g (96% of theory) of 3-chloro-5-(3,3-dichloroallyloxy)-2-methoxybenzoic acid.

Melting point: 103° C.

¹H-NMR (300 MHz, CDCl₃): δ (ppm)=4.02 (s, 3H); 4.66 (d, 2H); 6.14 (t, 1H); 7.20 (d, 1H); 7.53 (d, 1H).

### Step 6

### Preparation of [3-chloro-5-(3,3-dichloroallyloxy)-2-methoxyphenyl]methanol

Under nitrogen as protective gas, 3.1 g (10.0 mmol) of 3-chloro-5-(3,3-dichloroallyloxy)-2-methoxybenzoic acid are dissolved in about 30 ml of dry THF, and 15 ml of a 1M solution of borane (15 mmol) in THF are added with stirring. After the evolution of hydrogen has ceased, the mixture is allowed to stand at room temperature for about 18 hours. For work-up, initially about 10 ml of water are added with stirring to destroy excess borane, about 20 ml of dilute aqueous sodium hydroxide solution are then added and the mixture is extracted twice with in each case 10 ml of heptane. Concentration of the organic phase affords a pale yellow oil.

This gives 2.55 g (86% of theory) of [3-chloro-5-(3,3-dichloroallyloxy)-2-methoxyphenyl]-methanol.

¹H-NMR (300 MHz, CDCl₃): δ (ppm)=2.16 (t, 1H); 3.86 (s, 3H); 4.61 (d, 2H); 4.71 (d, 2H); 6.14 (t, 1H); 6.86 (s, 2H).

### Step 7

### Preparation of 3-chloro-5-(3,3-dichloroallyloxy)-2-methoxybenzaldehyde

Under nitrogen as protective gas, 2.5 g (8.5 mmol) of [3-chloro-5-(3,3-dichloroallyloxy)-2-methoxyphenyl]methanol are initially charged in about 50 ml of dry dichloromethane, and 2.2 g (10.2 mmol) of pyridinium chlorochromate (PCC) are added with stirring. Even shortly after the addition, the color of the reaction solution changes to dark brown. The suspension is allowed to stir for another 2 hours approximately, about 5 ml of isopropanol are then added to scavenge excess PCC and the mixture is stirred for about 10 minutes. For work-up, the mixture is filtered through a pleated filter, the residue is washed with dichloromethane and the brown filtrate is concentrated to about 10 ml and applied to a filter column (about 150 g of silica gel “conditioned” with dichloromethane; mobile phase: dichloromethane).

Concentration of the eluate to dryness gives 2.05 g (82% of theory) of 3-chloro-5-(3,3-dichloroallyloxy)-2-methoxybenzaldehyde as a colorless solid.

Melting point: 78° C.

¹H-NMR (300 MHz, CDCl₃): δ (ppm)=3.96 (s, 3H); 4.66 (d, 2H); 6.14 (t, 1H); 7.23 (s, 2H); 10.33 (s, 1H).

### Step 8

### Preparation of 3-chloro-5-(3,3-dichloroallyloxy)-2-methoxybenzaldehyde oxime

2.7 g (9.14 mmol) of 3-chloro-5-(3,3-dichloroallyloxy)-2-methoxybenzaldehyde, 0.7 g (10.0 mmol) of hydroxylammonium chloride and 0.82 g (10.0 mmol) of sodium acetate are suspended in a mixture of 30 ml of ethanol and 15 ml of water and, with stirring, heated to 50° C. for about one hour. The ethanol is then removed under reduced pressure on a rotary evaporator, and the aqueous suspension that remains is extracted with dichloromethane.

Concentration of the organic phase to dryness gives 2.55 g (90% of theory) of 3-chloro-5-(3,3-dichloroallyloxy)-2-methoxybenzaldehyde oxime as a colorless solid.

Melting point: 111° C.

MS (ES+): 310

¹H-NMR (400 MHz, CDCl₃): δ (ppm)=3.83 (s, 3H); 4.64 (d, 2H); 6.13 (t, 1H); 6.99 (d, 1H); 7.20 (d, 1H); 7.56 (bs, 1H); 8.37 (s, 1H).

### Further Starting Materials:

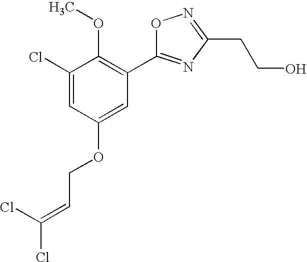

### Preparation of 2-{5-[3-chloro-5-(3,3-dichloroallyloxy)-2-methoxyphenyl]-1,2,4-oxadiazol-3-yl}-ethanol

134 mg (0.41 mmol) of O-(7-azabenzotriazol-1-yl)-N,N,N,N-tetramethyluronium PF6 (HATU), 13 mg (0.09 mmol) of 1-hydroxy-1H-benzotriazole hydrate (HOBT) and 82 mg (0.64 mmol) of N,N-diisopropylethylamine are added to 100 mg (0.32 mmol) of 3-chloro-5-(3,3-dichloroallyloxy)-2-methoxybenzoic acid in 5 ml of DMF. The mixture is stirred for 15 minutes. 100 mg (0.96 mmol) of 3,N-dihydroxypropionamidine are then added, and the mixture is stirred at room temperature for about 18 hours. About 10 ml of water are added, the mixture is extracted repeatedly with dichloromethane (DCM) and the organic phase is dried over sodium sulfate, filtered and concentrated to dryness.

For dehydration, the residue is taken up in 5 ml of DMF and, under a gentle stream of nitrogen, heated at 110° C. for 6 hours. The mixture is allowed to cool, diluted with DCM and washed with water, and the organic phase is dried over sodium sulfate and concentrated to dryness. Since (according to LC-MS) the conversion into the oxadiazole system was only 10%, the crude product obtained in this manner was once more subjected to the same reaction conditions.

This gives 120 mg (21% of theory according to LC-MS) of 2-{5-[3-chloro-5-(3,3-dichloroallyloxy)-2-methoxyphenyl]-1,2,4-oxadiazol-3-yl}ethanol which can be used for subsequent reactions.

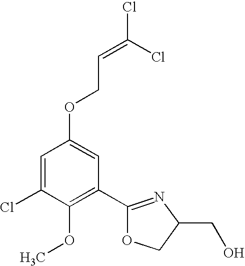

### Preparation of {2-[3-chloro-5-(3,3-dichloroallyloxy)-2-methoxyphenyl]-4,5-dihydrooxazol-4-yl}-methanol

Under nitrogen as protective gas, 200 mg of 3-chloro-5-(3,3-dichloroallyloxy)-2-methoxybenzamide are dissolved in 5 ml of absolute 1,2-dichloroethane, and 141 mg of triethyloxonium tetrafluoroborate are added. The mixture is stirred at room temperature overnight, and the solid slowly goes into solution. 2-Aminopropane-1,3-diol is added dropwise as a solution in 2 ml of dichloroethane, and the reaction mixture is stirred at room temperature for another 48 hours. 10 ml of saturated NaHCO₃ solution are then added. The aqueous phase is extracted repeatedly with dichloromethane (DCM), dried over sodium sulfate, filtered and concentrated to dryness. The crude product is chromatographed on silica gel (DCM:methanol 95:5).

This gives 103 mg (77% of theory according to LC-MS, 33% of theory) of {2-[3-chloro-5-(3,3-dichloroallyloxy)-2-methoxyphenyl]-4,5-dihydrooxazol-4-yl}methanol

MS (ES+)=366

log P (pH=2.3): 2.05

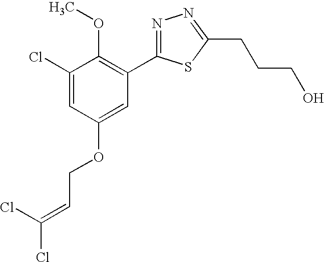

### Preparation of 3-{5-[3-chloro-5-(3,3-dichloroallyloxy)-2-methoxyphenyl]-[1,3,4]thiadiazol-2-yl}-propan-1-ol

a) 5 g of methyl 3-chloro-5-(3,3-dichloroallyloxy)-2-methoxybenzoate and 2.3 g of hydrazine hydrate in 30 ml of ethanol are stirred under reflux for 16 hours. The reaction mixture is then concentrated to dryness under reduced pressure. This gives 5.1 g (88% of theory) of 3-chloro-5-(3,3-dichloroallyloxy)-2-methoxybenzoic acid hydrazide in 87% purity of 87%.

MS (ES+)=325

b) 880 mg of 3-chloro-5-(3,3-dichloroallyloxy)-2-methoxybenzoic acid hydrazide are dissolved in 50 ml of dioxane. At 10° C., 1.15 g of 4-benzyloxybutyryl chloride are then added, and the mixture is stirred at room temperature for 18 hours. The reaction mixture is then added to water and extracted with ethyl acetate. The organic phase is removed and concentrated to dryness. This gives 1.3 g (79% of theory) of 3-chloro-5-(3,3-dichloroallyloxy)-2-methoxybenzoic acid N′-(4-benzyloxybutyryl)hydrazide in a purity of 82%.

| | |
|---|---|
| MS (ES+) = 501 | log P (pH = 2.3): 3.79 |

c) 1.3 g of 3-chloro-5-(3,3-dichloroallyloxy)-2-methoxybenzoic acid N′-(4-benzyloxybutyryl)hydrazide and 576 mg of Lawesson's reagent in 50 ml of toluene are stirred under reflux for 16 hours. The reaction mixture is then partitioned between water and toluene. The organic phase is separated off and concentrated to dryness. This gives 1.28 g (65% of theory) of 2-(3-benzyloxypropyl)-5-[3-chloro-5-(3,3-dichloroallyloxy)-2-methoxyphenyl]-[1,3,4]thiadiazole in a purity of 66%.

| | |
|---|---|
| MS (ES+) = 501 | log P (pH = 2.3): 5.67 |

d) 1.0 g of 2-(3-benzyloxypropyl)-5-[3-chloro-5-(3,3-dichloroallyloxy)-2-methoxyphenyl]-[1,3,4]thiadiazole, 2.27 g of boron trifluoride etherate and 620 mg of ethanethiol in 20 ml of dichloromethane are stirred under reflux for 16 hours. The reaction mixture is then added to water and extracted with ethyl acetate. The organic phase is separated off and concentrated to dryness. The residue that remains is purified on a silica gel column (silica gel 60—Merck, particle size: 0.04 to 0.063 mm) using the mobile phase ethyl acetate:hexane. This gives 320 mg (37% of theory) of 3-{5-[3-chloro-5-(3,3-dichloroallyloxy)-2-methoxyphenyl]-[1,3,4]thiadiazol-2-yl}propan-1-ol in a purity of 94%.

MS (ES+)=411

### 4-(1-Penten-5-yloxy)benzaldehyde

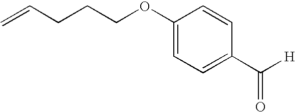

a) 5.0 g (58.0 mmol) of 4-penten-1-ol are initially charged in 50 ml of toluene, 3.0 g (30.4 mmol) of triethylamine are added and 6.6 g (58.0 mmol) of methanesulfonyl chloride are added dropwise at 0-5° C. The entire reaction mixture is then stirred at room temperature for 1.5 hours. The reaction mixture is then added to 240 ml of water and stirred for about 30 minutes. The organic phase is separated off, washed with water and dried. After concentration under reduced pressure, the crude product that remains is purified on a silica gel column (silica gel 60—Merck, particle size: 0.04 to 0.063 mm) using the mobile phase cyclohexane:acetone (8:1). This gives 3.9 g (41.2% of theory) of O-methylsulfonyl-1-pentenol.

¹H-NMR (CD₃CN, 400 MHz) δ=1.80-1.82 (br, 2H, —CH₂—), 1.92-1.95 (br., 2H, —CH₂—),

2.99 (s, 3H, —O—SO₂—CH₃); 4.20 (t, 2H, —O—CH₂—); 4.99-5.10 (br, 2H, ═CH2); 5.80-5.90 (br. 1H, —CH═) ppm.

b) 743.6 mg (6.1 mmol) of 4-hydroxybenzaldehyde and 1.00 g (6.1 mmol) of O-methylsulfonyl-1-pentenol are stirred in 60 ml of N,N-dimethylformamide, and 1.68 g (12.2 mmol) of potassium carbonate are added. The reaction mixture is then stirred at room temperature for 96 hours. For work-up, the mixture is stirred with 10% strength hydrochloric acid and extracted with toluene. Removal of the organic solvent under reduced pressure gives 961 mg (82.1% of theory) of 4-(1-penten-5-yloxy)benzaldehyde which can be used for the subsequent cycloaddition.

| | |
|---|---|
| LC-MS (ESI positive) m/e = 191 (MH⁺) | C₁₂H₁₄O₂ (190.2) |

The compounds listed below in table 1 can be obtained in an analogous manner.

TABLE 1

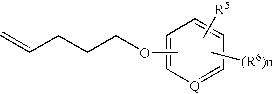

| No. | 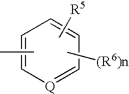 | analytical data*⁾ |
|---|---|---|
| A | 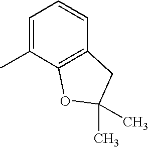 | m/e = 232 (M⁺) |
| B | 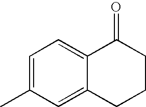 | m/e = 231 (MH⁺) |

*⁾LC-MS (ESI positive)

### Example A

### Spodoptera frugiperda Test (Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of corn leaves (Zea mays) are sprayed with an active compound preparation of the desired concentration and, after drying, populated with caterpillars of the armyworm (Spodoptera frugiperda).

After the desired period of time, the effect in % is determined. 100% means that all caterpillars have been killed; 0% means that none of the caterpillars have been killed.

In this test, for example, the compound of Preparation Example 1 shows good activity:

TABLE A
Plant-damaging insects
Spodoptera frugiperda test

| | Active | |
|---|---|---|
| | compound | Kill rate |
| | concentration | in % |
| Active compounds | in g/ha | after 7^{d} |
| 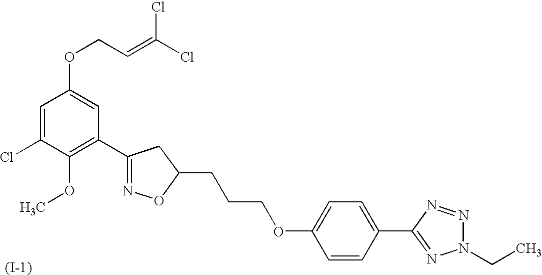 | 500 | 100 |
| 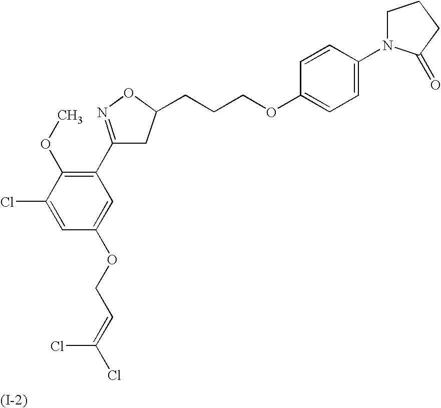 | 500 | 100 |
| 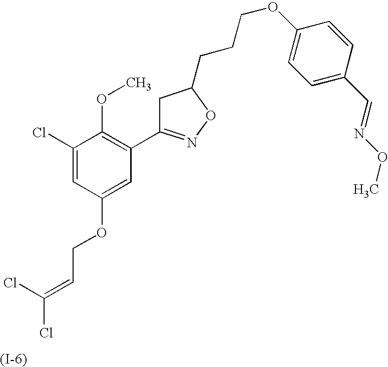 | 500 | 100 |
| 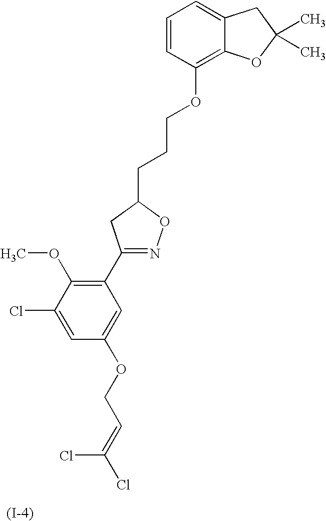 | 500 | 100 |
| 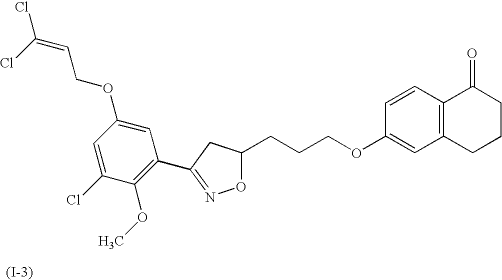 | 500 | 100 |

### Example B

### Tetranychus Test; OP-Resistant (TETRUR Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of bean leaves (Phaseolus vulgaris) which are infested by all stages of the greenhouse red spider mite (Tetranychus urticae) are sprayed with an active compound preparation of the desired concentration.

After the desired period of time, the effect in % is determined. 100% means that all spider mites have been killed; 0% means that none of the spider mites have been killed.

In this test, for example, the following compound of the Preparation Examples shows good activity: see table B.

TABLE B
Plant-damaging insects
Tetranychus test

| | Active | |
|---|---|---|
| | compound | |
| | concentra- | Kill rate |
| | tion | in % |
| Active compounds | in g/ha | after 7^{d} |
| 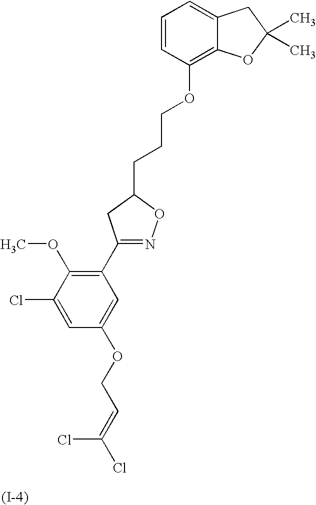 | 100 | 90 |

1. A compound of the formula (I), 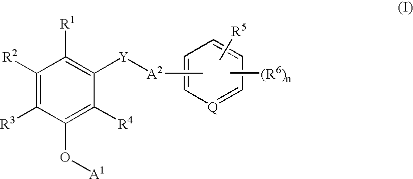
in which
n represents the number 0, 1 or 2,
A¹ represents one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃, —CH₂—CH═CClCH₃, where X represents halogen,
A² represents in each case straight-chain or branched alkanediyl or alkenediyl having in each case up to 8 carbon atoms which optionally contains at the beginning, at the end or within the carbon chain an oxygen atom, a sulfur atom or a grouping selected from the group consisting of SO, SO₂, NH and N(C₁-C₄-alkyl),
Q represents CH or N (nitrogen),
R¹ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino, alkylcarbonylamino or alkoximinoalkyl having in each case 1 to 10 carbon atoms in the alkyl groups, represents in each case optionally nitro-, hydroxyl-, amino-, cyano-, halogen-, C₁-C₆-alkyl-, C₁-C₆-haloalkyl-, C₁-C₆-alkoxy- or C₁-C₆-haloalkoxy-substituted aryloxy, arylthio or arylalkyl having in each case 6 or 10 carbon atoms in the aryl groups and, if appropriate, 1 to 4 carbon atoms in the alkyl moiety, represents in each case optionally nitro-, hydroxyl-, amino-, cyano-, halogen-, C₁-C₆-alkyl-, C₁-C₆-haloalkyl-, C₁-C₆-alkoxy- or C₁-C₆-haloalkoxy-substituted heterocyclyloxy or heterocyclylthio having in each case up to 10 carbon atoms, up to 4 nitrogen atoms and, if appropriate, one oxygen or sulfur atom, or represents the grouping —O-A¹, where A¹ is as defined above, or represents the grouping —N(R,R′), where R and R′ together represent straight-chain or branched alkanediyl having up to 8 carbon atoms which optionally contains at the beginning, at the end or within the carbon chain an oxygen atom, a sulfur atom or a grouping selected from the group consisting of SO, SO₂, NH and N(C₁-C₄-alkyl),
R² represents hydrogen, nitro, hydroxyl, amino, cyano, cyanato, thiocyanato, formyl, halogen, represents in each case optionally cyano-, halogen- or C₁-C₆-alkoxy-substituted alkyl, alkoxy, alkylthio, alkylsulfinyl, alkylsulfonyl, alkylamino, dialkylamino or alkylcarbonylamino having in each case 1 to 6 carbon atoms in the alkyl groups, represents C₁-C₆-alkyl-carbonyl, C₁-C₆-alkoxy-carbonyl, C₁-C₆-alkoximinoformyl, C₁-C₆-alkoximino-acetyl, or represents C₂-C₆-alkenyl or C₂-C₆-alkynyl,
R³ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁴ represents hydrogen, nitro, hydroxyl, amino, cyano, halogen, represents in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁵ represents an alkenyl grouping or alkynyl grouping having in each case 2 to 6 carbon atoms or a cycloalkenyl grouping having 4 to 6 carbon atoms, each of which groupings contains at least one substituent from the group consisting of nitro, cyano, carboxyl, carbamoyl, hydroxyl, carbonyl (C═O), hydroximino (C═N—OH), C₁-C₆-alkoxy, C₁-C₆-alkoxy-carbonyl, C₁-C₆-alkylamino, di-(C₁-C₆-alkyl)-amino, C₁-C₆-alkylamino-carbonyl, C₁-C₆-alkoxy-carbonylamino, C₁-C₆-alkoxy-C₁-C₆-alkoxy, C₁-C₆-alkoxyimino, C₃-C₆-alkenyloxy, C₃-C₆-alkynyloxy, C₃-C₆-alkenyloxy-carbonyl, C₃-C₆-alkynyloxy-carbonyl, C₃-C₆-alkenyloxyimino, C₃-C₆-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl, or
R⁵ represents a grouping -A³-Z, where
A³ represents a single bond or represents straight-chain or branched alkanediyl having 1 to 6 carbon atoms which is optionally substituted by halogen or C₃-C₆-cycloalkyl and
z represents monocyclic or bicyclic heteroaryl having up to 10 carbon atoms and at least one heteroatom from the group consisting of N (nitrogen, 1 to 5 N atoms), O (oxygen, 1 or 2 O atoms), sulfur (1 or 2 S atoms) and optionally as a replacement or additionally an SO or SO₂ grouping and optionally additionally a carbonyl grouping (C═O) and/or a thiocarbonyl grouping (C═S) as component of the heterocycle, which heteroaryl is optionally substituted by nitro, hydroxyl, mercapto, amino, formyl, cyano, carboxyl, carbamoyl, halogen, C₁-C₆-alkyl, C₁-C₆-hydroxyalkyl, C₁-C₆-haloalkyl, C₁-C₆-alkyl-carbonyl, C₁-C₆-haloalkyl-carbonyl, C₁-C₆-alkoxy, C₁-C₆-hydroxyalkoxy, C₁-C₆-haloalkoxy, C₁-C₆-alkoxy-carbonyl, C₁-C₆-haloalkoxy-carbonyl, C₁-C₆-alkoxy-C₁-C₆-alkyl, C₁-C₆-haloalkoxy-C₁-C₆-alkyl, C₁-C₂-alkylenedioxy, C₁-C₂-haloalkylenedioxy, C₁-C₆-alkoxyimino-C₁-C₆-alkyl, C₂-C₆-alkenyl, C₂-C₆-alkenyl-carbonyl, C₂-C₆-haloalkenyl, C₂-C₆-haloalkenyl-carbonyl, C₂-C₆-alkenyloxy-C₁-C₆-alkyl, C₂-C₆-haloalkenyloxy-C₁-C₆-alkyl, C₂-C₆-alkynyl, C₂-C₆-haloalkynyl, C₂-C₆-alkenyloxy, C₂-C₆-alkenyloxy-carbonyl, C₂-C₆-haloalkenyloxy, C₂-C₆-haloalkenyloxy-carbonyl, C₂-C₆-alkynloxy, C₂-C₆-alkynyloxy-carbonyl, C₂-C₆-haloalkynyloxy, C₂-C₆-haloalkynyloxy-carbonyl, C₂-C₆-alkynyloxy-C₁-C₆-alkyl, C₂-C₆-haloalkynyloxy-C₁-C₆-alkyl, C₃-C₈-cycloalkyl, C₃-C₈-cycloalkyl-carbonyl, C₃-C₈-cycloalkyl-C₁-C₆-alkyl, C₃-C₈-cycloalkyl-C₁-C₆-alkyl-carbonyl, C₃-C₈-cycloalkyloxy, C₃-C₈-cycloalkyloxy-carbonyl, C₃-C₈-cycloalkyl-C₁-C₆-alkoxy, C₃-C₈-cycloalkyl-C₁-C₆-alkoxy-carbonyl, C₃-C₈-cycloalkyloxy-C₁-C₆-alkyl, C₃-C₈-cycloalkyloxy-C₁-C₆-alkoxy, C₃-C₈-cycloalkyl-C₁-C₆-alkoxy-C₁-C₆-alkyl, C₁-C₆-alkyl-carbonyl-C₁-C₆-alkyl, C₁-C₆-alkoxy-carbonyl-C₁-C₆-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₂-C₆-alkenylthio, C₂-C₆-haloalkenylthio, C₂-C₆-alkynylthio, C₃-C₆-cycloalkylthio, C₃-C₆-cycloalkyl-C₁-C₆-alkylthio, C₁-C₆-alkylamino, C₁-C₆-alkylamino-carbonyl, di(C₁-C₆-alkyl)-amino, di-(C₁-C₆-alkyl)-amino-carbonyl, C₁-C₆-alkylcarbonylamino, C₁-C₆-haloalkyl-carbonylamino, C₁-C₆-alkoxy-carbonylamino, C₁-C₆-alkyl-aminocarbonylamino, or by phenyl, phenyloxy, benzyl, benzyloxy, phenylamino, benzylamino (where in each case the phenyl groups are optionally substituted by nitro, hydroxyl, mercapto, amino, cyano, C₁-C₆-alkyl, C₁-C₆-haloalkyl, C₁-C₆-alkyl-carbonyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-haloalkenyloxy, C₂-C₆-alkynyloxy, C₁-C₆-alkylthio, C₁-C₆-alkylsulfinyl, C₁-C₆-alkylsulfonyl or C₁-C₆-alkoxy-carbonyl),
R⁶ represents nitro, hydroxyl, mercapto, amino, formyl, cyano, carboxyl, carbamoyl, halogen, C₁-C₆-alkyl, C₁-C₆-hydroxyalkyl, C₁-C₆-haloalkyl, C₁-C₆-alkyl-carbonyl, C₁-C₆-haloalkyl-carbonyl, C₁-C₆-alkoxy, C₁-C₆-hydroxyalkoxy, C₁-C₆-haloalkoxy, C₁-C₆-alkoxy-carbonyl, C₁-C₆-haloalkoxy-carbonyl, C₁-C₆-alkoxy-C₁-C₆-alkyl, C₁-C₆-haloalkoxy-C₁-C₆-alkyl, C₁-C₆-alkoxyimino-C₁-C₆-alkyl, C₂-C₆-alkenyl, C₂-C₆-alkenyl-carbonyl, C₂-C₆-haloalkenyl, C₂-C₆-haloalkenyl-carbonyl, C₂-C₆-alkenyloxy-C₁-C₆-alkyl, C₂-C₆-haloalkenyloxy-C₁-C₆-alkyl, C₂-C₆-alkynyl, C₂-C₆-haloalkynyl, C₂-C₆-alkenyloxy, C₂-C₆-alkenyloxy-carbonyl, C₂-C₆-haloalkenyloxy, C₂-C₆-haloalkenyloxy-carbonyl, C₂-C₆-alkynyloxy, C₂-C₆-alkynyloxy-carbonyl, C₂-C₆-haloalkynyloxy, C₂-C₆-haloalkynyloxy-carbonyl, C₂-C₆-alkynyloxy-C₁-C₆-alkyl, C₂-C₆-haloalkynyloxy-C₁-C₆-alkyl, C₁-C₆-alkyl-carbonyl-C₁-C₆-alkyl, C₁-C₆-alkoxy-carbonyl-C₁-C₆-alkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₂-C₆-alkenylthio, C₂-C₆-haloalkenylthio, C₂-C₆-alkynylthio, C₁-C₆-alkylamino, C₁-C₆-alkylamino-carbonyl, di-(C₁-C₆-alkyl)-amino, di-(C₁-C₆-alkyl)-amino-carbonyl, C₁-C₆-alkyl-carbonylamino, C₁-C₆-haloalkyl-carbonylamino, C₁-C₆-alkoxy-carbonylamino or C₁-C₆-alkyl-aminocarbonylamino, and
Y represents a five- or six-membered heterocyclic grouping having at least 2 carbon atoms, at least one nitrogen atom and optionally one oxygen or sulfur atom, which grouping is attached in two different positions to the adjacent groupings, represents in particular a heterocyclic grouping selected from the list below (here, the exocyclic bonds indicate in each case the linkages to the adjacent groupings according to the arrangement in formula (I)), 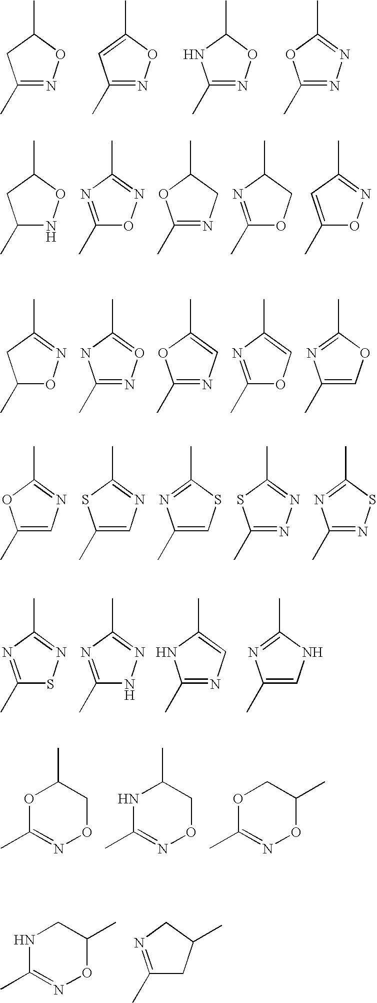
where these heterocyclic groupings may in each case optionally be substituted by one or two substituents from the group consisting of nitro, hydroxyl, amino, cyano, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio.

2. The compound of the formula (I) as claimed in claim 1, characterized in that
n represents the number 0, 1 or 2,
A¹ represents one of the groupings below
—CH₂—CH═CCl₂, —CH₂—CH═CBr₂, —CH₂—CH═CClF, —CH₂—CH═CBrCl, —CH₂—CH═CBrF,
A² represents in each case straight-chain or branched alkanediyl or alkenediyl having in each case up to 4 carbon atoms which optionally contains at the beginning, at the end or within the carbon chain an oxygen atom, a sulfur atom or a grouping selected from the group consisting of SO, SO₂, NH and N(C₁-C₃-alkyl),
Q represents CH or N (nitrogen),
R¹ represents hydrogen, nitro, hydroxyl, amino, cyano, halogen, represents in each case optionally cyano-, halogen- or C₁-C₅-alkoxy-substituted alkyl, alkoxy, alkylthio, alkylamino, dialkylamino, alkylcarbonylamino or alkoximinoalkyl having in each case 1 to 8 carbon atoms in the alkyl groups, represents in each case optionally nitro-, hydroxyl-, amino-, cyano-, halogen-, C₁-C₅-alkyl-, C₁-C₅-haloalkyl-, C₁-C₅-alkoxy- or C₁-C₅-haloalkoxy-substituted aryloxy, arylthio or arylalkyl having in each case 6 or 10 carbon atoms in the aryl groups and, if appropriate, 1 to 3 carbon atoms in the alkyl moiety, represents in each case optionally nitro-, hydroxyl-, amino-, cyano-, halogen-, C₁-C₅-alkyl-, C₁-C₅-haloalkyl-, C₁-C₅-alkoxy- or C₁-C₅-haloalkoxy-substituted heterocyclyloxy or heterocyclylthio having in each case up to 9 carbon atoms, 1 to 4 nitrogen atoms and/or one oxygen or sulfur atom, or represents the grouping —O-A¹, where A¹ is as defined above, or represents the grouping —N(R,R′), where R and R′ together represent straight-chain or branched alkanediyl having up to 6 carbon atoms which optionally contains at the beginning, at the end or within the carbon chain an oxygen atom, a sulfur atom or a grouping selected from the group consisting of SO, SO₂, NH and N(C₁-C₃-alkyl),
R² represents hydrogen, nitro, cyano, cyanato, thiocyanato, formyl, halogen, represents in each case optionally cyano-, halogen- or C₁-C₅-alkoxy-substituted alkyl, alkoxy, alkylthio, alkylamino, dialkylamino or alkylcarbonylamino having in each case 1 to 5 carbon atoms in the alkyl groups, represents C₁-C₅-alkyl-carbonyl, C₁-C₅-alkoxy-carbonyl, C₁-C₅-alkoximinoformyl, C₁-C₅-alkoximino-acetyl, or represents C₂-C₅-alkenyl or C₂-C₅-alkynyl,
R³ represents hydrogen, nitro, halogen, represents in each case optionally cyano-, halogen- or C₁-C₅-alkoxy-substituted alkyl, alkoxy, alkylthio or alkylamino having in each case 1 to 5 carbon atoms in the alkyl groups,
R⁴ represents hydrogen, nitro, halogen, represents in each case optionally cyano-, halogen- or C₁-C₅-alkoxy-substituted alkyl, alkoxy, alkylthio or alkylamino having in each case 1 to 5 carbon atoms in the alkyl groups,
R⁵ represents an alkenyl grouping or alkynyl grouping having in each case 2 to 5 carbon atoms or a cycloalkenyl grouping having 4 to 6 carbon atoms, each of which groupings contains at least one substituent from the group consisting of nitro, cyano, carboxyl, carbamoyl, hydroxyl, carbonyl (C═O), hydroximino (C═N—OH), C₁-C₅-alkoxy, C₁-C₅-alkoxy-carbonyl, C₁-C₅-alkylamino, di-(C₁-C₄-alkyl)-amino, C₁-C₅-alkylamino-carbonyl, C₁-C₅-alkoxy-carbonylamino, C₁-C₅-alkoxy-C₁-C₅-alkoxy, C₁-C₅-alkoxyimino, C₃-C₅-alkenyloxy, C₃-C₅-alkynyloxy, C₃-C₅-alkenyloxy-carbonyl, C₃-C₅-alkynyloxy-carbonyl, C₃-C₅-alkenyloxyimino, C₃-C₅-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl,
R⁵ furthermore represents a grouping -A³-Z, where
A³ represents a single bond or represents straight-chain or branched alkanediyl having 1 to 5 carbon atoms which is optionally substituted by halogen or C₃-C₆-cycloalkyl and
Z represents monocyclic or bicyclic heteroaryl having up to 9 carbon atoms and at least one heteroatom from the group consisting of N (nitrogen, 1 to 5 N atoms), O (oxygen, 1 or 2 O atoms), sulfur (1 or 2 S atoms) and optionally as a replacement or additionally an SO or SO₂ grouping and optionally additionally a carbonyl grouping (C═O) and/or a thiocarbonyl grouping (C═S) as component of the heterocycle, which heteroaryl is optionally substituted by nitro, hydroxyl, mercapto, amino, formyl, cyano, carboxyl, carbamoyl, halogen, C₁-C₅-alkyl, C₁-C₅-hydroxyalkyl, C₁-C₅-haloalkyl, C₁-C₅-alkyl-carbonyl, C₁-C₅-haloalkyl-carbonyl, C₁-C₅-alkoxy, C₁-C₅-hydroxyalkoxy, C₁-C₅-haloalkoxy, C₁-C₅-alkoxy-carbonyl, C₁-C₅-haloalkoxy-carbonyl, C₁-C₅-alkoxy-C₁-C₅-alkyl, C₁-C₅-haloalkoxy-C₁-C₅-alkyl, C₁-C₂-alkylenedioxy, C₁-C₂-haloalkylenedioxy, C₁-C₅-alkoxyimino-C₁-C₅-alkyl, C₂-C₅-alkenyl, C₂-C₅-alkenyl-carbonyl, C₂-C₅-haloalkenyl, C₂-C₅-haloalkenyl-carbonyl, C₃-C₅-alkenyloxy-C₁-C₆-alkyl, C₃-C₅-haloalkenyloxy-C₁-C₅-alkyl, C₂-C₅-alkynyl, C₂-C₅-haloalkynyl, C₃-C₅-alkenyloxy, C₃-C₅-alkenyloxy-carbonyl, C₃-C₅-haloalkenyloxy, C₃-C₅-haloalkenyloxy-carbonyl, C₃-C₅-alkynyloxy, C₃-C₅-alkynyloxy-carbonyl, C₃-C₅-haloalkynyloxy, C₃-C₅-haloalkynyloxy-carbonyl, C₃-C₅-alkynyloxy-C₁-C₅-alkyl, C₃-C₅-haloalkynyloxy-C₁-C₅-alkyl, C₃-C₇-cycloalkyl, C₃-C₇-cycloalkyl-carbonyl, C₃-C₇-cycloalkyl-C₁-C₅-alkyl, C₃-C₇-cycloalkyl-C₁-C₅-alkyl-carbonyl, C₃-C₇-cycloalkyloxy, C₃-C₇-cycloalkyloxy-carbonyl, C₃-C₇-cycloalkyl-C₁-C₅-alkoxy, C₃-C₇-cycloalkyl-C₁-C₅-alkoxy-carbonyl, C₃-C₇-cycloalkyloxy-C₁-C₅-alkyl, C₃-C₇-cycloalkyloxy-C₁-C₅-alkoxy, C₃-C₇-cycloalkyl-C₁-C₅-alkoxy-C₁-C₅-alkyl, C₁-C₅-alkyl-carbonyl-C₁-C₅-alkyl, C₁-C₅-alkoxy-carbonyl-C₁-C₅-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, C₂-C₅-alkenylthio, C₂-C₅-haloalkenylthio, C₂-C₅-alkynylthio, C₃-C₆-cycloalkylthio, C₃-C₆-cycloalkyl-C₁-C₅-alkylthio, C₁-C₅-alkylamino, C₁-C₅-alkylamino-carbonyl, di-(C₁-C₄-alkyl)-amino, di-(C₁-C₄-alkyl)-amino-carbonyl, C₁-C₅-alkyl-carbonylamino, C₁-C₅-haloalkyl-carbonylamino, C₁-C₅-alkoxy-carbonylamino, C₁-C₅-alkyl-aminocarbonylamino, or by phenyl, phenyloxy, benzyl, benzyloxy, phenylamino, benzylamino (where the phenyl groups are in each case optionally substituted by nitro, hydroxyl, mercapto, amino, cyano, C₁-C₅-alkyl, C₁-C₅-haloalkyl, C₁-C₅-alkyl-carbonyl, C₂-C₅-alkenyl, C₂-C₅-haloalkenyl, C₂-C₅-alkynyl, C₂-C₅-haloalkynyl, C₁-C₅-alkoxy, C₁-C₅-haloalkoxy, C₂-C₅-alkenyloxy, C₂-C₅-haloalkenyloxy, C₂-C₅-alkynyloxy, C₁-C₅-alkylthio, C₁-C₅-alkylsulfinyl, C₁-C₅-alkylsulfonyl or C₁-C₅-alkoxy-carbonyl),
R⁶ represents nitro, hydroxyl, mercapto, amino, formyl, cyano, carboxyl, carbamoyl, halogen, C₁-C₅-alkyl, C₁-C₅-hydroxyalkyl, C₁-C₅-haloalkyl, C₁-C₅-alkyl-carbonyl, C₁-C₅-haloalkyl-carbonyl, C₁-C₅-alkoxy, C₁-C₅-hydroxyalkoxy, C₁-C₅-haloalkoxy, C₁-C₅-alkoxy-carbonyl, C₁-C₅-haloalkoxy-carbonyl, C₁-C₅-alkoxy-C₁-C₅-alkyl, C₁-C₅-haloalkoxy-C₁-C₅-alkyl, C₁-C₅-alkoxyimino-C₁-C₅-alkyl, C₂-C₅-alkenyl, C₂-C₅-alkenyl-carbonyl, C₂-C₅-haloalkenyl, C₂-C₅-haloalkenyl-carbonyl, C₂-C₅-alkenyloxy-C₁-C₅-alkyl, C₂-C₅-haloalkenyloxy-C₁-C₅-alkyl, C₂-C₅-alkynyl, C₂-C₅-haloalkynyl, C₂-C₅-alkenyloxy, C₃-C₅-alkenyloxy-carbonyl, C₃-C₅-haloalkenyloxy, C₃-C₅-haloalkenyloxy-carbonyl, C₃-C₅-alkynyloxy, C₃-C₅-alkynyloxy-carbonyl, C₃-C₅-haloalkynyloxy, C₃-C₅-haloalkynyloxy-carbonyl, C₃-C₅-alkynyloxy-C₁-C₆-alkyl, C₃-C₅-haloalkynyloxy-C₁-C₅-alkyl, C₁-C₅-alkyl-carbonyl-C₁-C₅-alkyl, C₁-C₅-alkoxy-carbonyl-C₁-C₅-alkyl, C₁-C₅-alkylthio, C₁-C₅-haloalkylthio, C₁-C₅-alkylsulfinyl, C₁-C₅-haloalkylsulfinyl, C₁-C₅-alkylsulfonyl, C₁-C₅-haloalkylsulfonyl, C₃-C₅-alkenylthio, C₃-C₅-haloalkenylthio, C₃-C₅-alkynylthio, C₁-C₅-alkylamino, C₁-C₅-alkylamino-carbonyl, di-(C₁-C₄-alkyl)-amino, di-(C₁-C₄-alkyl)-amino-carbonyl, C₁-C₅-alkyl-carbonylamino, C₁-C₅-haloalkyl-carbonylamino, C₁-C₅-alkoxy-carbonylamino or C₁-C₅-alkyl-aminocarbonylamino,
Y represents a heterocyclic grouping which is attached in two different positions to the adjacent groupings and selected from the following list (here, the exocyclic bonds indicate in each case the linkages to the adjacent groupings according to the arrangement in formula (I)), 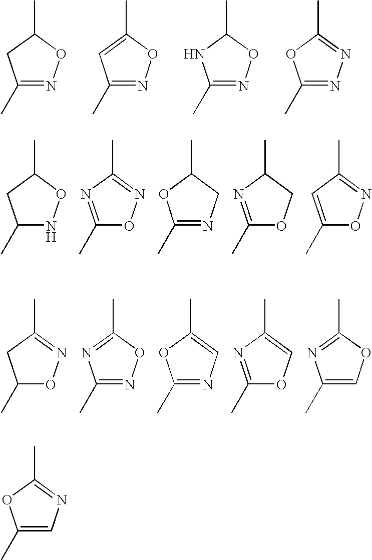
where these heterocyclic groupings may in each case optionally be substituted by one or two substituents from the group consisting of nitro, hydroxyl, amino, cyano, halogen, C₁-C₅-alkyl, C₁-C₅-haloalkyl, C₁-C₅-alkoxy, C₁-C₅-haloalkoxy, C₁-C₅-alkylthio, C₁-C₅-haloalkylthio.

3. The compound of the formula (I) as claimed in claim 1, characterized in that
n represents the number 0 or 1,
A¹ represents one of the groupings below:
—CH₂—CH═CCl₂, —CH₂—CH═CBr₂, —CH₂—CH═CBrCl,
A² represents one of the alkanediyl groupings listed below
—CH₂—, —CH₂CH₂—, —CH(CH₃)—CH₂—, —CH₂CH(CH₃)—, —CH₂CH₂CH₂—, —CH(CH₃)CH₂CH₂—, —CH₂CH(CH₃)CH₂—, —CH₂CH₂CH(CH₃)—, —CH₂CH₂CH₂CH₂—, —CH₂CH₂CH₂CH₂CH₂—,
each of which optionally contains at the beginning, at the end or within the carbon chain an oxygen atom, a sulfur atom or a grouping selected from the group consisting of SO, SO₂, NH and N(methyl),
Q represents CH or N (nitrogen),
R¹ represents hydrogen, nitro, hydroxyl, amino, cyano, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, dipropylamino, acetylamino, propionylamino, n- or i-butyroylamino, methoximinomethyl, ethoximinomethyl, methoximinoethyl or ethoximinoethyl, represents in each case optionally nitro-, hydroxyl-, amino-, cyano-, fluorine-, chlorine-, bromine-, iodine-, methyl-, ethyl-, n- or i-propyl-, n-, i-, s- or t-butyl-, difluoromethyl-, trifluoromethyl-, chlorodifluoromethyl-, fluoroethyl-, difluoroethyl-, trifluoroethyl-, chloroethyl-, dichloroethyl-, trichloroethyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted phenoxy, naphthyloxy, phenylthio, naphthylthio, benzyl or phenylethyl, represents in each case optionally nitro-, hydroxyl-, amino-, cyano-, fluorine-, chlorine-, bromine-, iodine-, methyl-, ethyl-, n- or i-propyl-, n-, i-, s- or t-butyl-, difluoromethyl-, trifluoromethyl-, chlorodifluoromethyl-, fluoroethyl-, difluoroethyl-, trifluoroethyl-, chloroethyl-, dichloroethyl-, trichloroethyl-, methoxy-, ethoxy-, n- or i-propoxy-, n-, i-, s- or t-butoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted heterocyclyloxy or heterocyclylthio having in each case up to 9 carbon atoms, 1 to 4 nitrogen atoms and/or one oxygen or sulfur atom, or represents the grouping —O-A¹, where A¹ is as defined above, or represents the grouping —N(R,R′) where R and R′ together with the N atom to which they are attached represent pyrrolidinyl, piperidinyl, morpholinyl or piperazinyl, each of which is optionally mono- or disubstituted by methyl and/or ethyl,
R² represents hydrogen, nitro, cyano, cyanato, thiocyanato, formyl, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, acetylamino, propionylamino, n- or i-butyroylamino, acetyl, propionyl, n- or i-butyroyl, methoxycarbonyl, ethoxycarbonyl, n- or i-propoxycarbonyl, methoximinoformyl, ethoximinoformyl, methoximinoacetyl or ethoximinoacetyl,
R³ represents hydrogen, nitro, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino,
R⁴ represents hydrogen, nitro, fluorine, chlorine, bromine, iodine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino,
R⁵ represents an ethenyl, propenyl, butenyl, pentenyl, ethynyl, propynyl, butynyl, pentynyl, cyclobutenyl, cyclopentenyl or cyclohexenyl grouping, each of which contains at least one substituent from the group consisting of nitro, cyano, carboxyl, carbamoyl, hydroxyl, carbonyl (C═O), hydroximino (C═N—OH), methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, methylamino, ethylamino, n- or i-propylamino, n-, i-, s- or t-butylamino, dimethylamino, diethylamino, dipropylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methoxymethoxy, ethoxymethoxy, n- or i-propoxymethoxy, n-, i-, s- or t-butoxymethoxy, methoxyethoxy, ethoxyethoxy, n- or i-propoxyethoxy, methoxypropoxy, ethoxypropoxy, methoxyimino, ethoxyimino, n- or i-propoxyimino, n-, i-, s- or t-butoxyimino, propenyloxy, butenyloxy, propynyloxy, butynyloxy, propenyloxycarbonyl, butenyloxycarbonyl, propynyloxycarbonyl, butynyloxycarbonyl, propenyloxyimino, butenyloxyimino, propynyloxyimino, butynyloxyimino, cyclopropyl, cyclobutyl, cyclopentyl, cyclohexyl, furyl, benzofuryl, thienyl, benzothienyl, isoxazolyl, benzisoxazolyl, pyrazolyl, pyridinyl, pyrimidinyl, pyrazinyl, pyridazinyl,
R⁵ furthermore represents a grouping -A³-Z, where
A³ represents a single bond or represents in each case optionally fluorine-, chlorine-, bromine-, cyclopropyl-, cyclobutyl-, cyclopentyl- or cyclohexyl-substituted methylene, ethane-1,1-diyl (ethylidene), ethane-1,2-diyl (dimethylene), propane-1,1-diyl (propylidene), propane-1,2-diyl, propane-1,3-diyl (trimethylene), butane-1,1-diyl (butylidene) or butane-1,4-diyl (tetramethylene) and
Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, hydroxy-n-propyl, hydroxy-i-propyl, fluoromethyl, chloromethyl, bromomethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-i-propyl, difluoro-i-propyl, trifluoro-i-propyl, tetrafluoro-i-propyl, pentafluoro-i-propyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, hydroxyethoxy, hydroxypropoxy, hydroxybutoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, difluoropropoxy, trifluoropropoxy, methoxycarbonyl, ethoxycarbonyl, n- or i-propoxycarbonyl, n-, i-, s- or t-butoxycarbonyl, fluoroethoxycarbonyl, chloroethoxycarbonyl, methoxymethyl, ethoxymethyl, n- or i-propoxymethyl, n-, i-, s- or t-butoxymethyl, methoxyethyl, ethoxyethyl, n- or i-propoxyethyl, fluoromethoxymethyl, difluoromethoxymethyl, fluoromethoxyethyl, difluoromethoxyethyl, methylenedioxy, dimethylenedioxy, difluoromethylenedioxy, difluorodimethylenedioxy, trifluorodimethylenedioxy, methoxyiminomethyl, ethoxyiminomethyl, n- or i-propoxyiminomethyl, n-, i-, s- or t-butoxyiminomethyl, methoxyiminoethyl, ethoxyiminoethyl, n- or i-propoxyiminoethyl, ethenyl, propenyl, butenyl, ethenylcarbonyl, propenylcarbonyl, butenylcarbonyl, fluoroethenyl, chloroethenyl, difluoroethenyl, dichloroethenyl, trifluoroethenyl, trichloroethenyl, fluoroethenylcarbonyl, chloroethenylcarbonyl, difluoroethenylcarbonyl, dichloroethenylcarbonyl, trifluoroethenylcarbonyl, trichloroethenylcarbonyl, propenyloxymethyl, butenyloxymethyl, propenyloxyethyl, butenyloxyethyl, fluoropropenyloxymethyl, chloropropenyloxymethyl, difluoropropenyloxymethyl, dichloropropenyloxymethyl, fluoropropenyloxyethyl, chloropropenyloxyethyl, difluoropropenyloxyethyl, dichloropropenyloxyethyl, ethynyl, propynyl, butynyl, fluoropropynyl, chloropropynyl, fluorobutynyl, chlorobutynyl, propenyloxy, butenyloxy, fluoropropenyloxy, chloropropenyloxy, fluorobutenyloxy, chlorobutenyloxy, propenyloxycarbonyl, butenyloxycarbonyl, fluoropropenyloxycarbonyl, chloropropenyloxycarbonyl, fluorobutenyloxycarbonyl, chlorobutenyloxycarbonyl, propynyloxy, butynyloxy, propynyloxycarbonyl, butynyloxycarbonyl, fluoropropynyloxy, chloropropynyloxy, fluorobutynyloxy, chlorobutynyloxy, fluoropropynyloxycarbonyl, chloropropynyloxycarbonyl, fluorobutynyloxycarbonyl, chlorobutynyloxycarbonyl, propynyloxymethyl, butynyloxymethyl, propynyloxyethyl, butynyloxyethyl, fluoropropynyloxymethyl, chloropropynyloxymethyl, fluorobutynyloxymethyl, chlorobutynyloxymethyl, fluoropropynyloxyethyl, chloropropynyloxyethyl, fluorobutynyloxyethyl, chlorobutynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, n-, i-, s- or t-butylthio, difluoromethylthio, trifluoromethylthio, chlorodifluoromethylthio, methylsulfinyl, ethylsulfinyl, n- or i-propylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, dimethylamino, diethylamino, dipropylamino, dibutylamino, dimethylaminocarbonyl, diethylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, methoxycarbonylamino, ethoxycarbonylamino, n- or i-propoxycarbonylamino, n-, i-, s- or t-butoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, acetyl, propionyl, n- or i-butyroyl, ethenyl, propenyl, butenyl, fluoroethenyl, chloroethenyl, difluoroethenyl, dichloroethenyl, trifluoroethenyl, trichloroethenyl, ethynyl, propynyl, butynyl, fluoropropynyl, chloropropynyl, fluorobutynyl, chlorobutynyl, methoxy, ethoxy, n- or i-propoxy, n-, i-, s- or t-butoxy, fluoromethoxy, difluoromethoxy, trifluoromethoxy, fluoroethoxy, difluoroethoxy, trifluoroethoxy, propenyloxy, butenyloxy, fluoropropenyloxy, chloropropenyloxy, fluorobutenyloxy, chlorobutenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or i-propoxycarbonyl, n-, i-, s- or t-butoxycarbonyl),
R⁶ represents nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, bromomethyl, difluoromethyl, dichloromethyl, trifluoromethyl, trichloromethyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, fluoromethoxy, difluoromethoxy, trifluoromethoxy, fluoroethoxy, chloroethoxy, difluoroethoxy, dichloroethoxy, trifluoroethoxy, fluoropropoxy, hydroxyethoxy, hydroxypropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, n-, i- s- or t-butoxymethyl, methoxyethyl, ethoxyethyl, n- or i-propoxyethyl, fluoromethoxymethyl, difluoromethoxymethyl, fluoromethoxyethyl, difluoromethoxyethyl, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, butenyl, ethenylcarbonyl, propenylcarbonyl, butenylcarbonyl, fluoroethenyl, chloroethenyl, difluoroethenyl, dichloroethenyl, trifluoroethenyl, fluoroethenylcarbonyl, chloroethenylcarbonyl, difluoroethenylcarbonyl, dichloroethenylcarbonyl, trifluoroethenylcarbonyl, propenyloxymethyl, butenyloxymethyl, propenyloxyethyl, butenyloxyethyl, fluoropropenyloxymethyl, chloropropenyloxymethyl, fluorobutenyloxymethyl, chlorobutenyloxymethyl, fluoropropenyloxyethyl, chloropropenyloxyethyl, fluorobutenyloxyethyl, chlorobutenyloxyethyl, ethynyl, propynyl, butynyl, fluoropropynyl, chloropropynyl, fluorobutynyl, chlorobutynyl, propenyloxy, butenyloxy, propenyloxycarbonyl, butenyloxycarbonyl, fluoropropenyloxy, chloropropenyloxy, fluorobutenyloxy, chlorobutenyloxy, fluoropropenyloxycarbonyl, chloropropenyloxycarbonyl, fluorobutenyloxycarbonyl, chlorobutenyloxycarbonyl, propynyloxy, butynyloxy, propynyloxycarbonyl, butynyloxycarbonyl, fluoropropynyloxy, chloropropynyloxy, fluorobutynyloxy, chlorobutynyloxy, fluoropropynyloxycarbonyl, chloropropynyloxycarbonyl, fluorobutynyloxycarbonyl, chlorobutynyloxycarbonyl, propynyloxymethyl, butynyloxymethyl, propynyloxyethyl, butynyloxyethyl, fluoropropynyloxymethyl, chloropropynyloxymethyl, fluorobutynyloxymethyl, chlorobutynyloxymethyl, acetylmethyl, propionylmethyl, n- or i-butyroylmethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, n-, i-, s- or t-butylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, dimethylamino, diethylamino, dipropylamino, dimethylaminocarbonyl, diethylaminocarbonyl, dipropylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, fluoropropionylamino, chloropropionylamino, methoxycarbonylamino, ethoxycarbonylamino, n- or i-propoxycarbonylamino, methylaminocarbonylamino, ethylaminocarbonylamino, n- or i-propylaminocarbonylamino,
Y represents a heterocyclic grouping which is attached in two different positions to the adjacent groupings and selected from the list below (here, the exocyclic bonds indicate in each case the linkages to the adjacent groupings according to the arrangement in formula (I)) 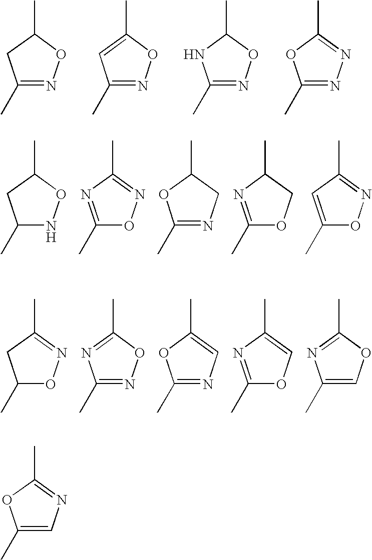
where these heterocyclic groupings may each optionally be substituted by one or two substituents from the group consisting of nitro, hydroxyl, amino, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, n-, i-, s- or t-butylthio, difluoromethylthio, trifluoromethylthio or chlorodifluoromethylthio.

4. The compound of the formula (I) as claimed in claim 1, characterized in that
n represents the number 0 or 1,
A¹ represents the grouping —CH₂—CH═CCl₂,
A² represents one of the groupings listed below:
—CH₂O—, —CH₂CH₂O—, —CH₂CH₂CH₂O—, —CH₂CH₂CH₂CH₂O—,
Q represents CH,
R¹ represents hydrogen, nitro, hydroxyl, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino, dimethylamino, represents in each case optionally nitro-, hydroxyl-, cyano-, fluorine-, chlorine-, bromine-, methyl-, ethyl-, n- or i-propyl-, n-, i-, s- or t-butyl-, difluoromethyl-, trifluoromethyl-, chlorodifluoromethyl-, fluoroethyl-, difluoroethyl-, trifluoroethyl-, chloroethyl-, dichloroethyl-, trichloroethyl-, methoxy-, ethoxy-, n- or i-propoxy-, fluoromethoxy-, difluoromethoxy-, trifluoromethoxy-, chlorodifluoromethoxy-, fluoroethoxy-, difluoroethoxy-, trifluoroethoxy-, chloroethoxy- or dichloroethoxy-substituted phenoxy, phenylthio, benzyl or phenylethyl, or represents the grouping —O-A¹, where A¹ has one of the meanings given above,
R² represents hydrogen, cyano, fluorine, chlorine, bromine, methyl, ethyl, methoxy or ethoxy,
R³ represents hydrogen, cyano, fluorine, chlorine, bromine, methyl, ethyl, methoxy or ethoxy,
R⁴ represents hydrogen, cyano, fluorine, chlorine or bromine,
R⁵ represents an ethenyl, propenyl, butenyl, pentenyl, ethynyl, propynyl, butynyl, pentynyl, cyclobutenyl, cyclopentenyl or cyclohexenyl grouping, each of which contains at least one substituent from the group consisting of nitro, cyano, hydroxyl, carbonyl (C═O), hydroximino (C═N—OH), methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, dimethylamino, diethylamino, methylaminocarbonyl, ethylaminocarbonyl, n- or i-propylaminocarbonyl, methoxycarbonylamino, ethoxycarbonylamino, n- or i-propoxycarbonylamino, n-, i-, s- or t-butoxycarbonylamino, methoxymethoxy, ethoxymethoxy, n- or i-propoxymethoxy, methoxyethoxy, ethoxyethoxy, n- or i-propoxyethoxy, methoxypropoxy, ethoxypropoxy, methoxyimino, ethoxyimino, n- or i-propoxyimino, propenyloxy, butenyloxy, propynyloxy butynyloxy, propenyloxycarbonyl, butenyloxycarbonyl, propynyloxycarbonyl, butynyloxycarbonyl, propenyloxyimino, butenyloxyimino, propynyloxyimino, butynyloxyimino, cyclopropyl, cyclobutyl, cyclopentyl, cyclohexyl, furyl, benzofuryl, thienyl, pyridinyl,
R⁵ furthermore represents a grouping -A³-Z, where
A³ represents a single bond or represents in each case optionally fluorine-, chlorine-, cyclopropyl-, cyclobutyl-, cyclopentyl- or cyclohexyl-substituted methylene, ethane-1,1-diyl (ethylidene), ethane-1,2-diyl (dimethylene), propane-1,1-diyl (propylidene), propane-1,2-diyl or propane-1,3-diyl (trimethylene) and
Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-i-propyl, difluoro-i-propyl, trifluoro-i-propyl, tetrafluoro-i-propyl, pentafluoro-i-propyl, acetyl, propionyl, n- or i-butyroyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, n-, i-, s- or t-butoxy, hydroxyethoxy, hydroxypropoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, n-, i-, s- or t-butoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, fluoromethoxymethyl, difluoromethoxymethyl, fluoromethoxyethyl, difluoromethoxyethyl, methylenedioxy, dimethylenedioxy, difluoromethylenedioxy, difluorodimethylenedioxy, trifluorodimethylenedioxy, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, butenyl, propenylcarbonyl, butenylcarbonyl, fluoroethenyl, chloroethenyl, difluoroethenyl, dichloroethenyl, trifluoroethenyl, trichloroethenyl, propenyloxymethyl, butenyloxymethyl, propenyloxyethyl, butenyloxyethyl, fluoropropenyloxymethyl, chloropropenyloxymethyl, difluoropropenyloxymethyl, dichloropropenyloxymethyl, fluoropropenyloxyethyl, chloropropenyloxyethyl, difluoropropenyloxyethyl, dichloropropenyloxyethyl, ethynyl, propynyl, butynyl, fluoropropynyl, chloropropynyl, fluorobutynyl, chlorobutynyl, propenyloxy, butenyloxy, fluoropropenyloxy, chloropropenyloxy, fluorobutenyloxy, chlorobutenyloxy, propenyloxycarbonyl, butenyloxycarbonyl, propynyloxy, butynyloxy, propynyloxycarbonyl, butynyloxycarbonyl, propynyloxymethyl, butynyloxymethyl, propynyloxyethyl, butynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, n- or i-butyroylamino, fluoroacetylamino, chloroacetylamino, difluoroacetylamino, dichloroacetylamino, trifluoroacetylamino, trichloroacetylamino, methoxycarbonylamino, ethoxycarbonylamino, n- or i-propoxycarbonylamino, n-, i-, s- or t-butoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, acetyl, propionyl, ethenyl, propenyl, ethynyl, propynyl, methoxy, ethoxy, n- or i-propoxy, fluoromethoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or ipropoxycarbonyl),
R⁶ represents nitro, hydroxyl, mercapto, amino, formyl, cyano, carboxyl, carbamoyl, fluorine, chlorine, bromine, methyl, ethyl, n- or i-propyl, fluoromethyl, chloromethyl, bromomethyl, difluoromethyl, dichloromethyl, trifluoromethyl, trichloromethyl, acetyl, propionyl, fluoroacetyl, chloroacetyl, difluoroacetyl, dichloroacetyl, trifluoroacetyl, trichloroacetyl, fluoropropionyl, chloropropionyl, methoxy, ethoxy, n- or i-propoxy, fluoromethoxy, difluoromethoxy, trifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, methoxyiminomethyl, ethoxyiminomethyl, n- or i-propoxyiminomethyl, methoxyiminoethyl, ethoxyiminoethyl, n- or i-propoxyiminoethyl, ethenyl, propenyl, fluoroethenyl, chloroethenyl, difluoroethenyl, dichloroethenyl, trifluoroethenyl, ethynyl, propynyl, butynyl, propenyloxy, butenyloxy, propenyloxycarbonyl, butenyloxycarbonyl, fluoropropenyloxy, chloropropenyloxy, fluorobutenyloxy, chlorobutenyloxy, propynyloxy, butynyloxy, propynyloxycarbonyl, butynyloxycarbonyl, propynyloxymethyl, butynyloxymethyl, propynyloxyethyl, butynyloxyethyl, acetylmethyl, propionylmethyl, n- or i-butyroylmethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, propynylthio, butynylthio, methylamino, ethylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, methoxycarbonylamino, ethoxycarbonylamino, n- or i-propoxycarbonylamino, methylaminocarbonylamino, ethylaminocarbonylamino, n- or i-propylaminocarbonylamino,
Y represents one of the heterocyclic groupings below (here, the exocyclic bonds indicate in each case the linkages to the adjacent groupings according to the arrangement in formula (I)), 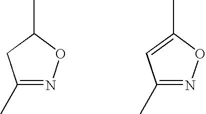
where each of these heterocyclic groupings may optionally be substituted by one or two substituents from the group consisting of nitro, hydroxyl, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, difluoromethylthio, trifluoromethylthio or chlorodifluoromethylthio.

5. The compound of the formula (I) as claimed in claim 1, characterized in that
R¹ represents hydrogen, nitro, hydroxyl, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino or dimethylamino,
R² represents hydrogen, fluorine, chlorine or bromine,
R⁵ represents a grouping -A³-Z, where
A³ represents a single bond or represents methylene and
Z represents monocyclic heteroaryl having up to 5 carbon atoms and at least one heteroatom from the group consisting of N (nitrogen, 1 to 4 N atoms), O (oxygen, 1 O atom), sulfur (1 S atom) and optionally as a replacement or additionally an SO or SO₂ grouping and optionally additionally a carbonyl grouping (C═O) and/or a thiocarbonyl grouping (C═S) as component of the heterocycle, where, as heteroaryl groupings, mention may be made in particular of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, very particularly however tetrazolyl, where each heteroaryl is optionally substituted by nitro, hydroxyl, mercapto, amino, formyl, cyano, carboxyl, carbamoyl, fluorine, chlorine, bromine, methyl, ethyl, n- or i-propyl, n-, i-, s- or t-butyl, hydroxymethyl, hydroxyethyl, fluoromethyl, chloromethyl, difluoromethyl, dichloromethyl, trifluoromethyl, trichloromethyl, fluoroethyl, chloroethyl, difluoroethyl, dichloroethyl, chlorofluoroethyl, trifluoroethyl, trichloroethyl, chlorodifluoroethyl, fluoropropyl, chloropropyl, difluoropropyl, dichloropropyl, trifluoropropyl, fluoro-i-propyl, difluoro-i-propyl, trifluoro-i-propyl, tetrafluoro-i-propyl, pentafluoro-i-propyl, methoxy, ethoxy, n- or i-propoxy, hydroxyethoxy, hydroxypropoxy, fluoromethoxy, difluoromethoxy, trifluoromethoxy, fluorodichloromethoxy, chlorodifluoromethoxy, fluoroethoxy, chloroethoxy, difluoroethoxy, dichloroethoxy, trifluoroethoxy, fluoropropoxy, methoxycarbonyl, ethoxycarbonyl, n- or i-propoxycarbonyl, fluoroethoxycarbonyl, chloroethoxycarbonyl, methoxymethyl, ethoxymethyl, n- or i-propoxymethyl, methoxyethyl, ethoxyethyl, n- or i-propoxyethyl, ethenyl, propenyl, butenyl, fluoroethenyl, chloroethenyl, difluoroethenyl, dichloroethenyl, trifluoroethenyl, trichloroethenyl, propenyloxymethyl, butenyloxymethyl, propenyloxyethyl, butenyloxyethyl, ethynyl, propynyl, butynyl, propenyloxy, butenyloxy, fluoropropenyloxy, chloropropenyloxy, fluorobutenyloxy, chlorobutenyloxy, propenyloxycarbonyl, butenyloxycarbonyl, propynyloxy, butynyloxy, propynyloxycarbonyl, butynyloxycarbonyl, propynyloxymethyl, butynyloxymethyl, propynyloxyethyl, butynyloxyethyl, cyclopropyl, cyclobutyl, cyclopentyl, cyclohexyl, cyclopropylcarbonyl, cyclobutylcarbonyl, cyclopentylcarbonyl, cyclohexylcarbonyl, cyclopropylmethyl, cyclobutylmethyl, cyclopentylmethyl, cyclohexylmethyl, cyclopropylmethylcarbonyl, cyclobutylmethylcarbonyl, cyclopentylmethylcarbonyl, cyclohexylmethylcarbonyl, cyclopropyloxy, cyclobutyloxy, cyclopentyloxy, cyclohexyloxy, cyclopropyloxycarbonyl, cyclobutyloxycarbonyl, cyclopentyloxycarbonyl, cyclohexyloxycarbonyl, cyclopropylmethoxy, cyclobutylmethoxy, cyclopentylmethoxy, cyclohexylmethoxy, cyclopropylmethoxycarbonyl, cyclobutylmethoxycarbonyl, cyclopentylmethoxycarbonyl, cyclohexylmethoxycarbonyl, cyclopropylmethoxymethyl, cyclobutylmethoxymethyl, cyclopentylmethoxymethyl, cyclohexylmethoxymethyl, cyclopropyloxymethoxy, cyclobutyloxymethoxy, cyclopentyloxymethoxy, cyclohexyloxymethoxy, acetylmethyl, propionylmethyl, n- or i-butyroylmethyl, acetylethyl, propionylethyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, methylthio, ethylthio, n- or i-propylthio, difluoromethylthio, trifluoromethylthio, chlorodifluoromethylthio, methylsulfinyl, ethylsulfinyl, trifluoromethylsulfinyl, methylsulfonyl, ethylsulfonyl, trifluoromethylsulfonyl, propenylthio, butenylthio, fluoropropenylthio, chloropropenylthio, fluorobutenylthio, chlorobutenylthio, propynylthio, butynylthio, cyclopropylthio, cyclobutylthio, cyclopentylthio, cyclohexylthio, cyclopropylmethylthio, cyclobutylmethylthio, cyclopentylmethylthio, cyclohexylmethylthio, methylamino, ethylamino, n- or i-propylamino, methylaminocarbonyl, ethylaminocarbonyl, n- or i-propylaminocarbonyl, dimethylamino, diethylamino, dimethylaminocarbonyl, diethylaminocarbonyl, acetylamino, propionylamino, n- or i-butyroylamino, methoxycarbonylamino, ethoxycarbonylamino, n- or i-propoxycarbonylamino, methylaminocarbonylamino, ethylaminocarbonylamino, n- or i-propylaminocarbonylamino, or by phenyl, phenyloxy, benzyl, benzyloxy, phenylamino, benzylamino (where the phenyl groups are in each case optionally substituted by nitro, hydroxyl, mercapto, amino, cyano, methyl, ethyl, n- or i-propyl, n-, i-, s- or t-butyl, trifluoromethyl, methoxy, ethoxy, n- or i-propoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or i-propoxycarbonyl),
Y represents one of the heterocyclic groupings below (here, the exocyclic bonds indicate in each case the linkages to the adjacent groupings according to the arrangement in formula (I)).

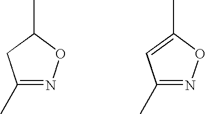

6. The compound of the formula (I) as claimed in claim 5, characterized in that
A¹ represents a grouping —CH₂—CH═Cl₂,
A² represents a grouping —CH₂—CH₂—CH₂—O—,
n has the value 1,
Q represents CH,
R¹ represents —CH₃,
R² represents chlorine,
R³, R⁴ and R⁶ represent hydrogen, 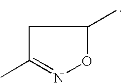
Y represents the grouping

7. A process for preparing compounds of the formula (I) as claimed in claim 1, characterized in that compounds of the formula (II) 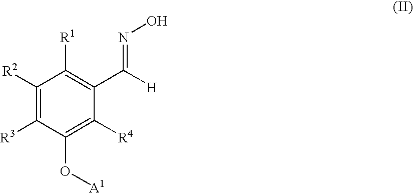
in which
A¹, R¹, R², R³ and R⁴ are as defined in claim 1
are reacted with halogenating agents, if appropriate in the presence of one or more diluents,
the compounds, formed in this reaction, of the general formula (III) 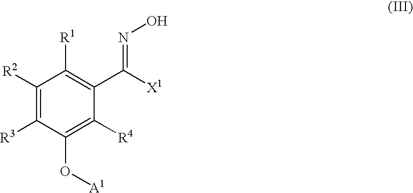
in which
A¹, R¹, R², R³ and R⁴ are as defined in claim 1 and
X¹ represents halogen
are reacted in situ with one or more acid binders,
and the compounds, formed in this manner, of the formula (IV) 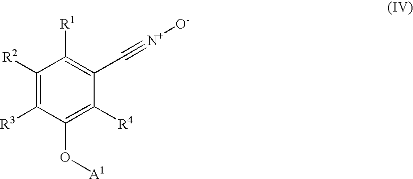
in which
A¹, R¹, R², R³ and R⁴ are as defined in claim 1
are reacted in situ with alkenes of the general formula (V) 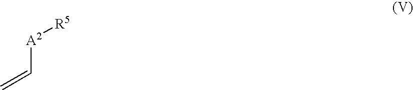 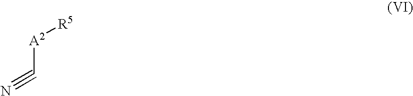
in which
A² and R⁵ are as defined in claim 1 and
for (V) the carbon atoms of the olefinic double bond are optionally substituted as indicated above for Y,
if appropriate in the presence of one or more diluents and if appropriate in the presence of one or more reaction auxiliaries,
and the compounds of the formula (I) obtained in this manner are, if appropriate, converted by customary methods into other compounds of the formula (I).

8. A compound of the formula (II), 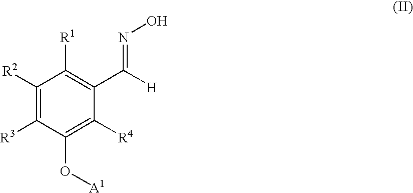
in which
A¹, R¹, R², R³ and R⁴ are as defined in claim 1.

9. A compound of the formula (VIII), 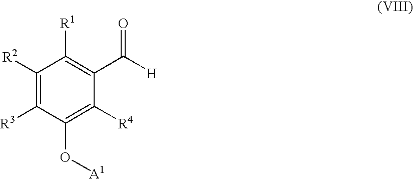
in which
A¹, R¹, R², R³ and R⁴ are as defined in claim 1,
except for the compound 3-[(3,3-dichloro-2-propenyl)oxy]benzaldehyde.

10. A compound of the formula (XI), 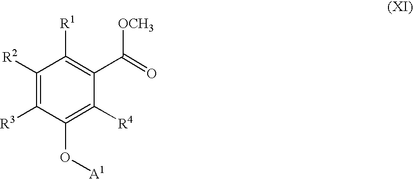
in which
A¹, R¹, R², R³ and R⁴ are as defined in claim 1.

11. A compound of the formula (XII), 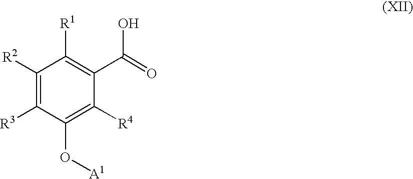
in which
A¹, R¹, R², R³ and R⁴ are as defined in claim 1.

12. A compound of the formula (XIII), 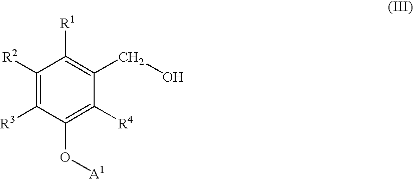
in which
A¹, R¹, R², R³ and R⁴ are as defined in claim 1.

13. A compound of the formula (XIV), 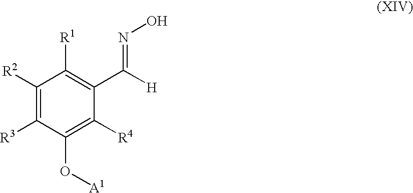
in which
A¹, R¹, R², R³ and R⁴ are as defined in claim 1.

14. A composition, characterized in that it comprises at least one compound of the formula (I) as claimed in claim 1 and customary extenders and/or surfactants.

15. A method for controlling pests, characterized in that a compound of the formula (I) as claimed in claim 1 or a composition as claimed in claim 14 is allowed to act on the pests and/or their habitat.

16. The use of compounds of the formula (I) as claimed in claim 1 or of compositions as claimed in claim 14 for controlling pests.
